(19) United States
(12) Patent Application Publication
(10) Pub. No.: US 2023/0242587 A1
(43) Pub. Date: Aug. 3, 2023

(54) THYCLOTIDES

(71) Applicant: The United States of America,as represented by the Secretary,Department of Health and Human Services, Bethesda, MD (US)

(72) Inventors: Daniel H. Appella, Rockville, MD (US); Hongchao Zheng, Bethesda, MD (US); Harsha C. Amarasekara, Rockville, MD (US); Victor Clausse, Bethesda, MD (US); George A. Kubi, Rockville, MD (US)

(73) Assignee: The United States of America,as represented by the Secretary,Department of Health and Human Services, Bethesda, MD (US)

(21) Appl. No.: 17/918,725

(22) PCT Filed: Apr. 15, 2021
(86) PCT No.: PCT/US2021/027397

§ 371 Date: Oct. 13, 2022

Related U.S. Application Data
(60) Provisional application No. 63/011,398, filed on Apr. 17, 2020.

(51) Int. Cl. C07K 14/00; C07D 401/12; C12N 15/113

(52) U.S. Cl. CPC C07K 14/003; C07D 401/12; C12N 15/113; C12N 2310/11

(57) ABSTRACT

Disclosed are thyclotides, which are oligomers, each comprising (a) from about 8 to about 25 monomer units of formula (I) and (b) from 0 to about 24 monomer units of formula (II): wherein B is a nucleobase, which can be the same or different at each occurrence, or a pharmaceutically acceptable salt thereof. The thyclotides are soluble in water, bind strongly to complementary DNA and RNA, and are cell permeable. The thyclotides are useful as reagents for antisense and antigene applications, and as probes in molecular diagnostics and microarrays.

### CROSS-REFERENCE TO A RELATED APPLICATION

This application claims the benefit of U.S. Provisional Patent Application No. 63/011,398, filed Apr. 17, 2020, the disclosure of which is incorporated herein by reference in its entirety for all purposes.

### FEDERALLY SPONSORED RESEARCH OR DEVELOPMENT

This invention was made with Government support under project number Z01-DK031143 by the National Institutes of Health, National Institute of Diabetes and Digestive and Kidney Diseases. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 1,063 Byte ASCII (Text) file named “752099_ST25.TXT,” created on Apr. 6, 2021.

### BACKGROUND OF THE INVENTION

Since the 1980s, there has been much focus on developing modified oligonucleotides for controlling gene expression, with the further purpose of being used as potential therapeutic agents for a wide variety of diseases (Uhlmann, E. et al., Chem. Rev. 90: 543 (1990); (Goodchild, J. Bioconjugate Chem. 1: 165 (1990); Deleavey, G. F. et al., J. Chem. Biol. 19: 937 (2012); Sharma, V. K. et al., Med. Chem. Commun. 5: 1454 (2014)). In 1991, Nielsen and co-workers were the first to describe one such class of nucleic acid analogues, peptide nucleic acids (PNAs) (Nielsen, P. E.; et al., Science 154: 1497 (1991); Egholm, M. et al., J Am. Chem. Soc. 114: 1895 (1992); Nielsen, P. E., Acc. Chem. Res. 32: 624 (1999); Nielsen, P. E., Curr. Opin. Biotechnol. 12: 16 (2001); Nielsen, P. E., Mol. Biotechnol. 26: 233 (2004). PNAs consist of nucleobases attached to an acyclic and highly flexible pseudopeptide backbone. Due to their uncharged and non-natural polyamide backbone, PNAs exhibits remarkably strong binding properties towards complementary oligonucleotides and high stability to enzyme degradation. These unique properties make PNAs ideal reagents for antisense and antigene applications, and as probes for molecular diagnostics and microarrays.

However, PNAs have some drawbacks such as poor cellular uptake and relatively low aqueous solubility. These inherent properties of PNAs have greatly limited their applications in biomedical areas. Numerous efforts have been made to modify the backbone of PNAs to address these concerns (Tedeschi, T. et al., Tetrahedron Lett. 46: 8395 (2005); Carradini, R. et al., Curr. Top. Med. Chem. 11: 1535 (2011); Rozners, E. J. Nucleic Acids No 518162 (2012); Sugiyama, T.; et al., Molecules 18: 287 (2013); Moccia, M. et al., Artif. DNA PNA XNA 5: No. e1107176 (2014). Although some of these chemical modifications have led to the improvement of water solubility and bio-compatibility, it was realized at the price of binding affinity and sequence specificity. In previous studies, it has been reported that introduction of non-polar cyclopentane rings could rigidify the backbone of PNAs (tcypPNAs 2) and therefore preorganize PNAs into right-handed helix forms to increase binding affinity to complementary nucleic acids (Myers, M. C. et al., Org. Lett. 5: 2695 (2003); Pokorski, J. K. et al., J Am. Chem. Soc. 126: 15067 (2004); Englund, E. A. et al., J Am. Chem. Soc. 128: 16456 (2006); (Micklitsch, C. M. et al., Anal. Chem. 85: 251 (2013); Zheng, H. et al., Org. Lett. 20: 7637 (2018)). Nevertheless, this backbone modification significantly decreases the polarity of PNAs and therefore results in worse water solubility.

Therefore, there remains an unmet need in the art to develop backbone-modified PNAs that could not only increase the binding properties to complementary nucleic acids, but also enhance the aqueous solubility and cellular uptake.

### BRIEF SUMMARY OF THE INVENTION

The invention provides an oligomer comprising (a) from about 8 to about 25 monomer units of formula (I):

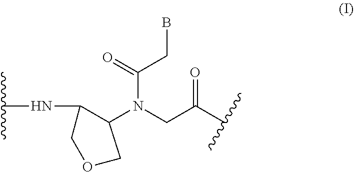

and
(b) from 0 to about 24 monomer units of formula (II):

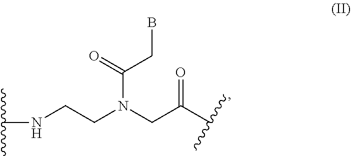

wherein B is a nucleobase, and wherein B can be the same or different at each occurrence,
   or a pharmaceutically acceptable salt thereof.

The invention also provides a pharmaceutical composition comprising a pharmaceutically acceptable carrier and an oligomer or salt of the invention.

The invention further provides a compound of formula (IV):

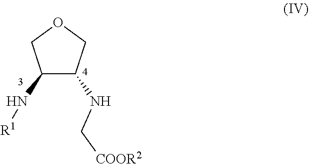

wherein R¹ is a nitrogen protecting group and wherein R² is selected from H, C₁-C₁₀ alkyl, C₆-C₁₀ aryl, and C₁-C₁₀ alkyl-C₆-C₁₀ aryl, wherein the configuration at positions 3 and 4 of the tetrahydrofuran ring is (3R,4R) or (3S,4S).

The invention additionally provides a compound of formula (V):

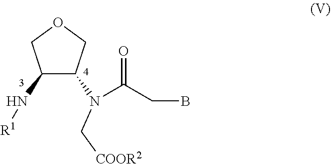

wherein R¹ is a nitrogen protecting group, wherein R² is selected from H, C₁-C₁₀ alkyl, C₆-C₁₀ aryl, and C₁-C₁₀ alkyl-C₆-C₁₀ aryl, and wherein B is an optionally protected nucleobase, wherein the configuration at positions 3 and 4 of the tetrahydrofuran ring is (3R,4R) or (3S,4S).

### BRIEF DESCRIPTION OF THE SEVERAL VIEWS OF THE DRAWING(S)

FIG. 1A shows the cell counts and scatter graphs as determined by flow cytometry for HCT116 cells treated with AEG-PNA in accordance with an aspect of the invention.

FIG. 1B shows the cell counts and scatter graphs as determined by flow cytometry for HCT116 cells treated with THF-PNA in accordance with an aspect of the invention.

FIG. 1C shows the cell counts and scatter graphs as determined by flow cytometry for Hep62 cells treated with AEG-PNA in accordance with an aspect of the invention.

FIG. 1D shows the cell counts and scatter graphs as determined by flow cytometry for Hep62 cells treated with THF-PNA in accordance with an aspect of the invention.

FIG. 2A shows the percent of positive cells for HCT116 cells treated with a AEG-PNA and a THF-PNA in accordance with an aspect of the invention.

FIG. 2B shows the mean fluorescence intensity (MFI) for HCT116 cells treated with a AEG-PNA and a THF-PNA in accordance with an aspect of the invention.

FIG. 2C shows the percent of positive cells for Hep62 cells treated with a AEG-PNA and a THF-PNA in accordance with an aspect of the invention.

FIG. 2D shows the mean fluorescence intensity (MFI) for Hep62 cells treated with a AEG-PNA and a THF-PNA in accordance with an aspect of the invention.

FIGS. 3A and 3B show microscopic images of HCT116 cells incubated with a AEG-PNA and a THF-PNA, respectively in accordance with an aspect of the invention.

FIG. 4A shows relative normalized miR-21 expression in cells incubated with 1 μM and 5 μM of a AEG-PNA in accordance with an aspect of the invention.

FIG. 4B shows normalized miR-21 expression as a function of concentration for cells treated with an anti-miR-21 THF-PNA in accordance with an aspect of the invention.

FIG. 4C shows the effect on normalized miR-21 expression for cells treated with an anti-miR-21 AEG-PNA as compared with a scramble control AEG-PNA in accordance with an aspect of the invention.

FIG. 5A shows the structures of an aegPNA and a THF-PNA, in accordance with an aspect of the invention. The right-hand N of the upper fragment is bonded to the left-hand C═O of the lower fragment for each oligomer and the broken bond is indicated by the perpendicular line in each fragment.

FIG. 5B shows the structures of SS-THF-PNA sequences in an aspect of the invention: (a) thiol-dPEG₂₄-A #C #T #T #T #G #A #G #A #C #T #A # and (b) thiol-dPEG₂₄-ACT #TTGAGACT #A. Base # represents Base-tcyp residue.

FIG. 6 shows the structures of the dyes fluorescein (as the isothiocyanate derivative thereof), Cy2, Cy3, Cy3.5, Cy5, Cy5.5, Cy7, and Cy7.5.

FIG. 7 shows overlaid HPLC chromatograms of an inventive oligomer, AEG-PNA, a mixed AEG and cyclopentyl PNA, and cyclopentyl-containing PNA in accordance with an aspect of the invention.

### DETAILED DESCRIPTION OF THE INVENTION

In an aspect, the invention provides an oligomer comprising (a) from about 8 to about 25 monomer units of formula (I):

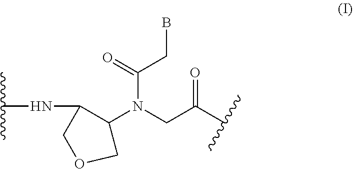

and
(b) from 0 to about 24 monomer units of formula (II):

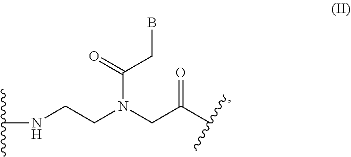

wherein B is a nucleobase, and wherein B can be the same or different at each occurrence,
   or a pharmaceutically acceptable salt thereof.

B can be any nucleobase and can be a natural nucleobase or a nucleobase of a non-natural nucleotide. Non-limiting examples of suitable natural nucleobase include adenine, thymine, uracil, guanine, and cytosine. Numerous nucleobases of non-natural nucleotides are known in the art. Some non-limiting examples of nucleobases of non-natural nucleotides include, for example, hypoxanthine, xanthine, 7-methylguanine, 5,6-dihydrouracil, 5-methylcytosine, 5-hydroxymethylcytosine, and pseudouridine. A readily available reference for modified RNAs and nucleobases and nucleotides contained therein is available. See Cantara, W. A. et al., Nucleic Acids Research, Vol. 39, Database issue D195-D201 (2011) and references described therein. The inventive oligomer is not limited to comprise any particular nucleobase, and any and all known nucleobases are included within the definition of the term nucleobase as used herein.

The inventive oligomers comprising a monomer unit of formula (I) are also referred to herein as thyclotides.

The oligomer comprises from about 8 to about 25 monomer units of formula (I) and from 0 to about 24 monomer units of formula (II). In preferred aspects, the oligonucleotide comprises a sum of from about 12 to about 25 monomer units of formula (I) and formula (II), e.g., the oligonucleotide comprises a sum of about 12, about 13, about 14, about 15, about 16, about 17, about 18, about 19, about 20, about 21, about 22, about 23, about 24, or about 25 monomer units of formula (I) and formula (II). In certain preferred aspects, the oligonucleotide comprises a sum of from about 15 to about 22 monomer units of formula (I) and formula (II).

In some aspects, the oligomer does not comprise a monomer unit of formula (II). In these aspects, the oligomer comprises a backbone that consists of only monomer units of formula (I). In some other aspects, the oligomer comprises a backbone that consists of only monomer units of formula (I) and formula (II).

As will be readily apparent to the ordinarily skilled person, the monomer of formula (I) has two chiral carbon atoms at positions 3 and 4 of the tetrahydrofuran ring:

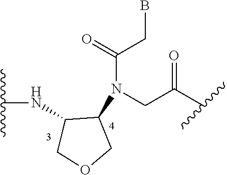

(depicted as a subunit within an oligomer). The 3- and 4-carbon atoms can have any configuration. Because the monomer of formula (I) has two chiral centers, the two chiral centers can form four diastereomeric pairs: (3R,4R), (3R,4S), (3S,4R), and (3S,4S). As drawn, the monomeric subunit of formula (I) has the (3R,4R) configuration. When the monomer of formula (I) has the (3R,4R) configuration, the monomer induces a right-handed helix into the PNA, which matches the right-handed helicity of DNA and RNA, thereby allowing for complex formation (by Watson-Crick binding) of the inventive oligomer and DNA or RNA. In an aspect of the invention, the monomer of formula (I) can be of (3S,4S) configuration.

In some aspects, the oligomer further comprises one or more monomer units of formula (III):

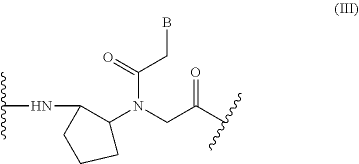

wherein B is a nucleobase, and wherein B can be the same or different at each occurrence.

As will be readily apparent to the ordinarily skilled person, the monomer of formula (III) has two chiral carbon atoms at positions 3 and 4 of the cyclopentyl ring:

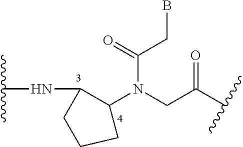

(depicted as a subunit within an oligomer). The 3- and 4-carbon atoms can have any configuration as discussed herein in connection the monomer unit of formula (I).

In certain of these aspects, the oligonucleotide comprises a sum of from about 12 to about 25 monomer units of formula (I), formula (II), and formula (III). In certain preferred aspects, the oligonucleotide comprises a sum of from about 15 to about 22 monomer units of formula (I), formula (II), and formula (III).

In any of these aspects, the oligomer has an amino terminus and a carboxy terminus, wherein the amino terminus is —NHR¹ wherein R¹ is H or a linker-attached group selected from dyes, radioimaging moieties, chelating moieties, nanoparticles, cytotoxic agents, and a second oligomer comprising (a) from about 8 to about 25 monomer units of formula (I) and (b) from 0 to about 24 monomer units of formula (II), wherein the monomer unit of formula (I) in the second oligomer has two chiral carbon atoms, wherein the two chiral carbon atoms of at least one monomer unit of formula (I) in the second oligomer have an (S,S) configuration.

The dye can be any dye suitable for use in biological applications. As used herein, the term “dye” refers to any moiety that is detectable by the use of electromagnetic radiation. The electromagnetic radiation can have a wavelength that is detectable by the human eye (visible light) or can have a wavelength within the ultraviolet range or infrared range, which can be detected by the use of suitable detectors. The dye can absorb electromagnetic radiation at a certain wavelength or narrow range or wavelengths, such that the electromagnetic radiation that is transmitted through a sample containing the dye will be depleted of the wavelength or range or wavelengths absorbed by the dye moiety. The dye moiety can be a fluorescent dye moiety (i.e., a fluorophore), which undergoes excitation by electromagnetic radiation impinging thereon and emits electromagnetic radiation of a different wavelength or range of wavelengths than the wavelength or range of wavelengths that causes the excitation. The dye moiety is not limited in any particular way and can be any suitable dye moiety useful for detection of the oligomer comprising the dye moiety. In certain aspects, R¹ comprises a dye selected from fluorescein, Cy2, Cy3, Cy3.5, Cy5, Cy5.5, Cy7, and Cy7.5.

The chelating moiety can be any suitable chelating moiety. As used herein, the term “chelating moiety” refers to a moiety that forms a chelate involving the formation or presence of two or more separate coordinate bonds between a polydentate (multiple bonded) ligand and a single central atom. Chelating moieties are also referred to as chelants, chelators, chelating agents, or sequestering agents. The chelating moiety can be any suitable chelating moiety that chelates any suitable ion or ions. Non-limiting examples of suitable ions include metal ions such as zinc (e.g., Zn²⁺), iron (e.g., Fe²⁺ and Fe³⁺), manganese, and the like. In certain aspects, R¹ comprises a chelating agent selected from 1,4,7-triazacyclononane-N,N′,N″-triacetic acid, 1,4,7,10-tetrazacyclononane-N,N′,N″-triacetic acid, triethylenetetramine, diethylenetetramine pentaacetic acid, and hydrazinonicotinamide. The chelating moiety can be covalently bonded to the linker by use of any functional group capable of forming a covalent bond with the chelating moiety. For example, 1,4,7-triazacyclononane-N,N′,N″-triacetic acid can be bonded to the linker by means of an amide group formed between one of the carboxylic acid groups on the chelating moiety and an amino or hydroxyl group on the linker by reaction of an active ester (e.g., mixed anhydride, succinimidoyl ester, HOBt ester, and the like) with the amino or hydroxyl group on the linker.

The radioimaging moiety can be any suitable moiety for use in magnetic resonance imaging, single photon emission imaging, positron emission tomographic imaging (PET), and single-photon emission computed tomography (SPECT). In some aspects, the radioimaging moiety comprises an isotope that emits radiation which can be detected using any suitable detection technique. Non-limiting examples of suitable isotopes include ¹⁸F-AlF, ⁶⁰Cu, ⁶¹Cu, ⁶²Cu, ⁶⁴Cu, ⁶⁷Cu, ⁶⁸Ga, ⁸⁶Y, ⁸⁹Zr, ¹¹¹In, ⁹⁹mTc, ¹⁸⁶Re, ¹⁸⁸Re, Gd³⁺, or Mn²⁺. The isotope is preferably present as a chelate within a suitable chelating moiety. The chelating moiety can be as described herein. In certain particular aspects, R¹ comprises a radioimaging agent selected from 1,4,7-triazacyclononane-N,N′,N″-triacetic acid, 1, 4, 7,10-tetrazacyclononane-N,N′,N″-triacetic acid, triethylenetetramine, diethylenetetramine pentaacetic acid, and hydrazinonicotinamide in combination with one or more of ¹⁸F-AlF, ⁶⁰Cu, ⁶¹Cu, ⁶²Cu, ⁶⁴Cu, ⁶⁷Cu, ⁶⁸Ga, ⁸⁶Y, ⁸⁹Zr, ¹¹¹In, ⁹⁹mTc, ¹⁸⁶Re, ¹⁸⁸Re, Gd³⁺, or Mn²⁺.

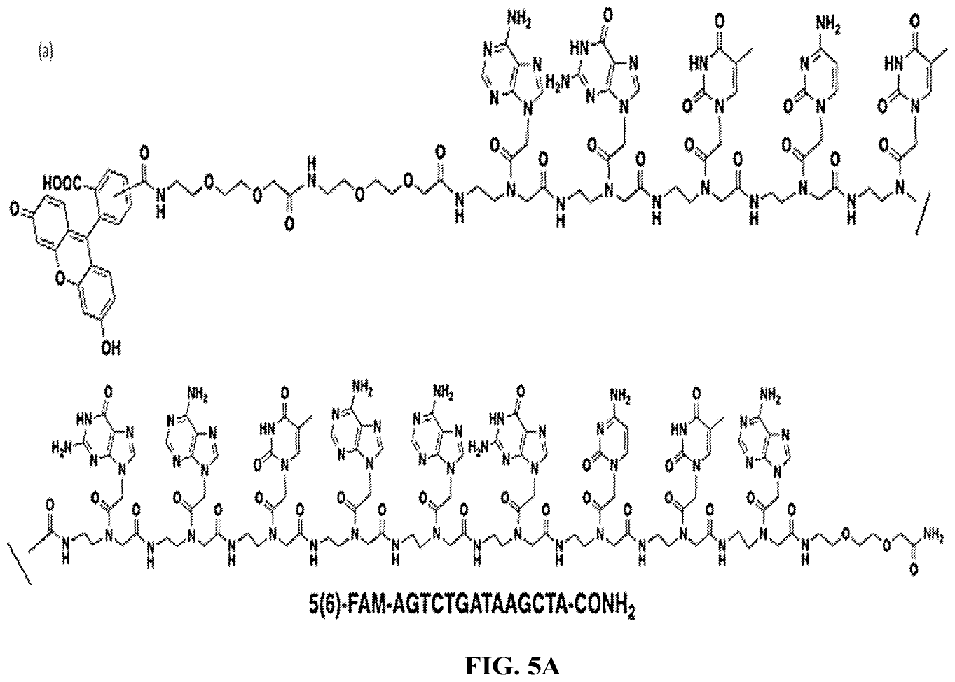

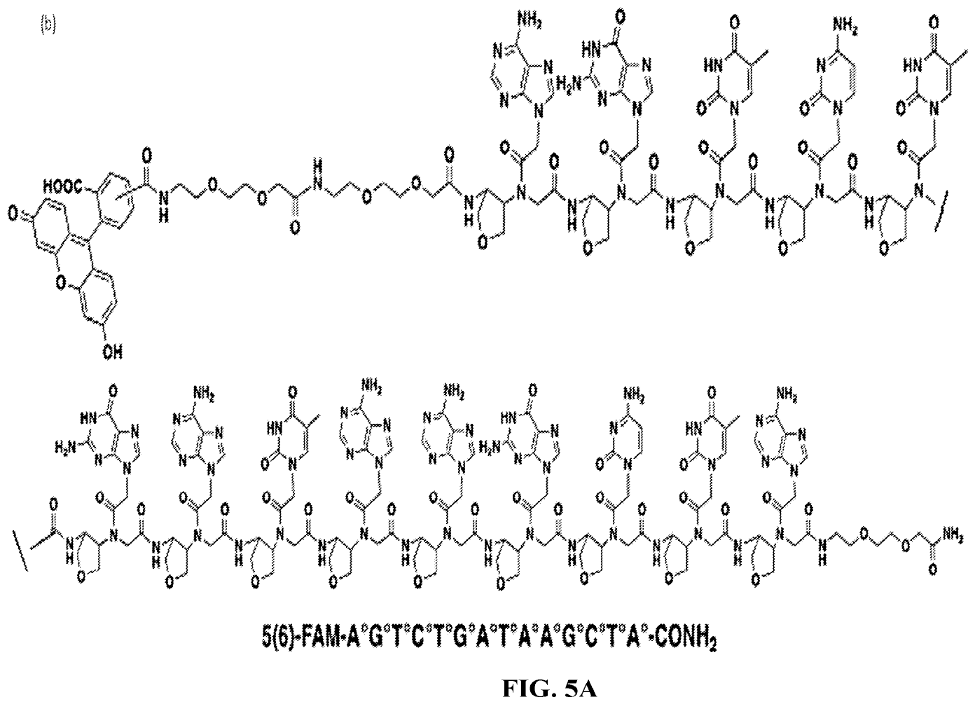

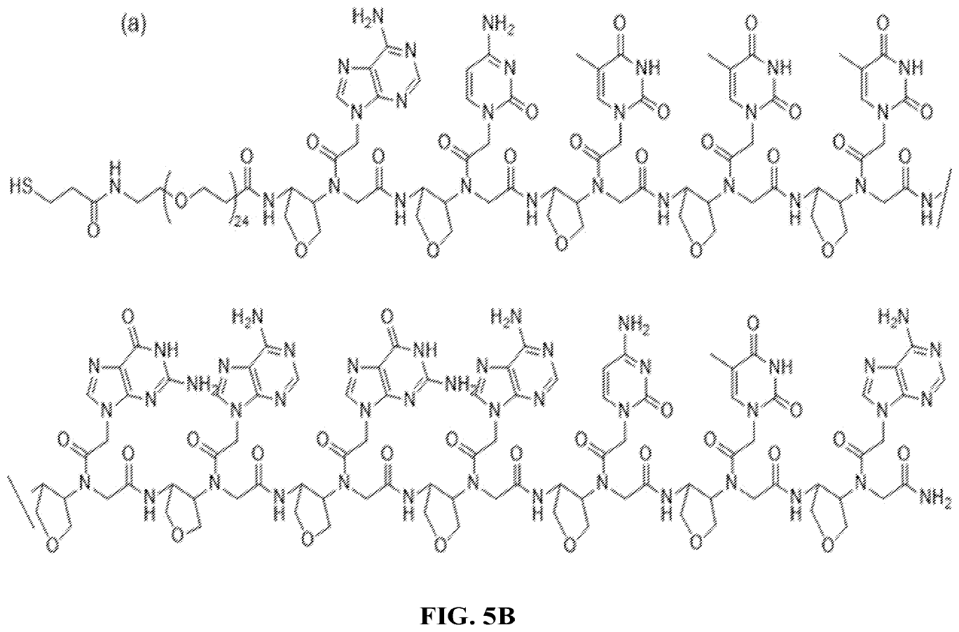

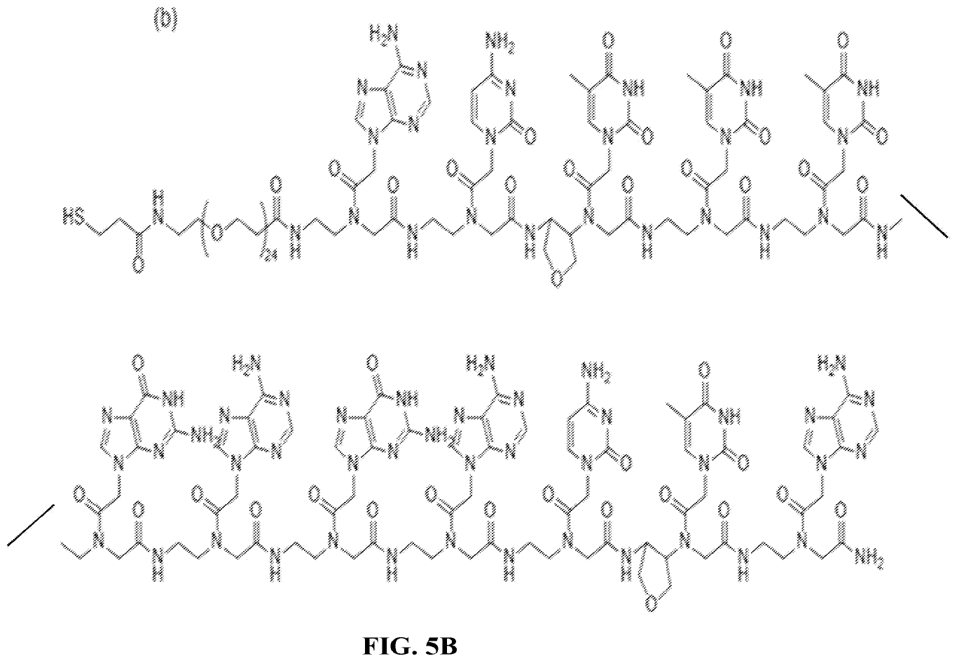

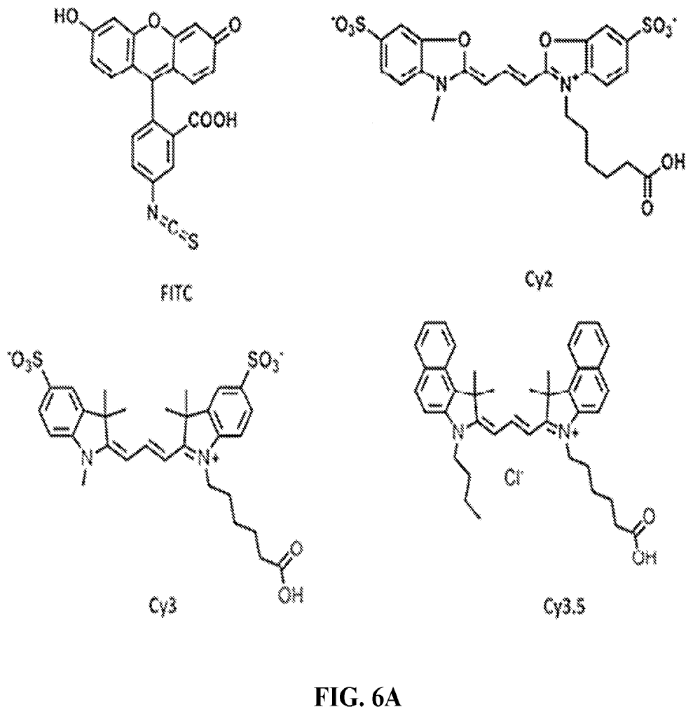

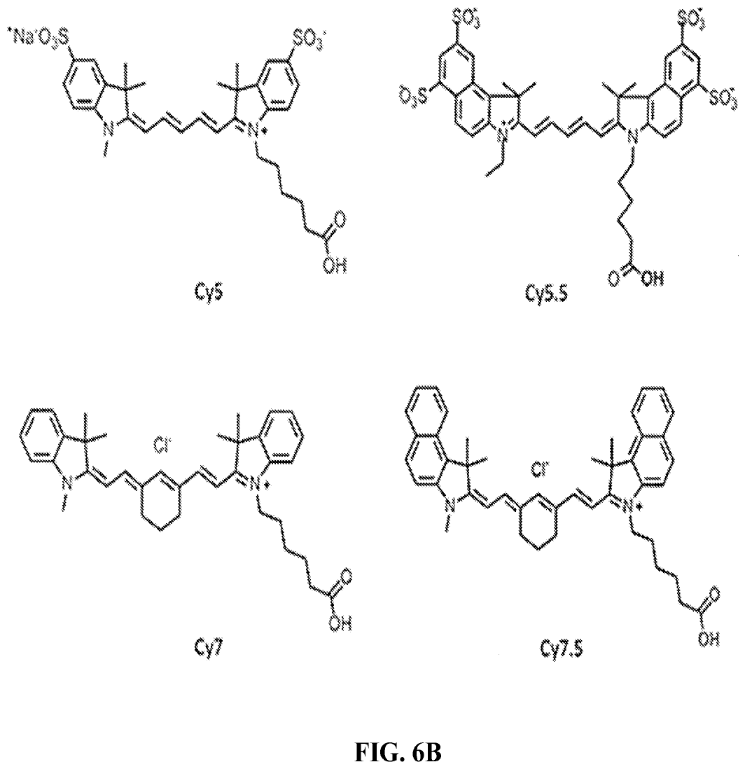

The nanoparticles can be any suitable nanoparticles. A non-limiting example of suitable nanoparticles is gold nanoparticles. The gold nanoparticles can be covalently or otherwise bonded to the linker via any suitable functional group. In a non-limiting example, the gold nanoparticles can be bonded via one or more coordinatively-bound sulfur atoms present on the linker. In a particular aspect, nanoparticles can be bonded to thiol moieties, for example, to the thiol moieties of sequences shown in FIG. 5B.

The cytotoxic agent can be any suitable cytotoxic agent. The anticancer agent can be chosen from reversible DNA binders, DNA alkylators, and DNA strand breakers.

Examples of suitable reversible DNA binders include topetecan hydrochloride, irinotecan (CPT11—Camptosar), rubitecan, exatecan, nalidixic acid, TAS-103, etoposide, acridines (such as amsacrine, aminocrine), actinomycins (such as actinomycin D), anthracyclines (such as doxorubicin, daunorubicin), benzophenainse, XR 11576/MLN 576, benzopyridoindoles, Mitoxantrone, AQ4, Etopside, Teniposide, (epipodophyllotoxins), and bisintercalating agents such as triostin A and echinomycin.

Examples of suitable DNA alkylators include sulfur mustard, the nitrogen mustards (such as mechlorethamine), chlorambucil, melphalan, ethyleneimines (such as triethylenemelamine, carboquone, diaziquone), methyl methanesulfonate, busulfan, CC-1065, duocarmycins (such as duocarmycin A, duocarmycin SA), metabolically activated alkylating agents such as nitrosoureas (such as carmustine, lomustine, (2-chloroethyl)nitrosoureas), triazine antitumor drugs such as triazenoimidazole (such as dacarbazine), mitomycin C, leinamycin, and the like.

Examples of suitable DNA strand breakers include doxorubicin and daunorubicin (which are also reversible DNA binders), other anthracyclines, bleomycins, tirapazamine, enediyne antitumor antibiotics such as neocarzinostatin, esperamicins, calicheamicins, dynemicin A, hedarcidin, C-1027, N1999A2, esperamicins, zinostatin, and the like.

The cytotoxic agent can be covalently bonded to the linker group by means of any suitable functional group capable of forming a covalent bond between the cytotoxic agent and the linker, for example, as described herein for bonding of dyes and chelating moieties to the linker.

R¹ can be a linker-attached second oligomer comprising (a) from about 8 to about 25 monomer units of formula (I) and (b) from 0 to about 24 monomer units of formula (II), wherein the monomer unit of formula (I) in the second oligomer has two chiral carbon atoms, wherein the two chiral carbon atoms of at least one monomer unit of formula (I) in the second oligomer have an (S,S) configuration. When the monomer of formula (I) has the (3S,4S) configuration, the monomer induces a left-handed helix into the PNA, which does not match the right-handed helicity of DNA and RNA, thereby inhibiting complex formation (by Watson-Crick binding) of the inventive oligomer and DNA or RNA. However, the second oligomer (having the (3S,4S)) configuration for the monomer of formula (I) can complex with a third oligomer also comprising monomers of formula (I) having the (3S,4S) configuration. Thus, an oligomer having an R¹ group comprising a linker attached second PNA oligomer comprising monomer units of formula (I) having the (3S,4S) configuration can bind to natural DNA or RNA at the oligomer portion comprising monomer units of formula (I) having the (3R,4R) configuration and further bind to a third PNA oligomer comprising monomer units of formula (I) having the (3S,4S) configuration, to form a tertiary complex.

The R¹ group comprises a linker attaching the linker-attached group. The linker can be any suitable linker and preferably the linker is covalently bound to the amino terminus nitrogen atom. The linker can comprise two or more functional groups that separately form a covalent bond to the amino terminus nitrogen atom and a covalent bond to the linker-attached group. Non-limiting examples of suitable functional groups for bonding to the amino terminus nitrogen atom include carbonyl (R—C(═O)—), ester (RO—C(═O)—), carbonate (RO—C(═O)O—), carbamate (RNR′—C(═O)—), thiocarbonyl (R—C(═S)—), dithioester (RS—C(═S)—), thiocarbamate (RNR′—C(═S)—), imino (RC═N—), and the like, to form the structures R—C(═O)—NH-oligomer, RO—C(═O)—NH-oligomer, (RNR′—C(═O)—NH-oligomer, R—C(═S)—NH-oligomer, RS—C(═S)—NH-oligomer, RNR′—C(═S)—NH-oligomer, RC═N-oligomer, respectively, and the like. Non-limiting examples of functional groups that form a covalent bond with the linker and the linker-attached moiety include nucleophilic groups such as N, O, and S, or the linker and the linker-attached moiety can be covalently bonded via a C—C single bond or C═C double bond. Many other suitable such functional groups will be readily known to the ordinarily skilled artisan. The linked can be covalently bound to the amino terminus of the oligonucleotide directly by a single or double bond without the intermediacy of a functional group, for example, by a direct C—N or C═N bond.

The linker can be covalently bound to the linker-attached group using any combination of functional groups that react to form a covalent bond, many of which will be understood by those of ordinary skill in the art. For example, if the linker-attached group has a carboxyl functional group, the carboxyl functional group can react with an amino group or a hydroxyl group on the linker to form an amide or ester bond connecting the linker-attached group to the linker.

The linker can be of length 1 to 120 atoms, and/or has one or some of the elements: C, N, O, S, P, and Si, and/or is in a chain that contains only one or a combination of the following bonds: a single bond, a double bond, a triple bond, an amide bond, an ester bond, a disulfide bond, an imino group, an ether bond, a thioether bond, and a thioester bond. For example, the linker can comprise a straight or branched chain C₁-C₂₀ alkyl, C₂-C₂₀ alkenyl, C₂-C₂₀ alkynyl, C₁-C₂₀ alkyl substituted within the chain with one or more heteroatoms selected from O and S, aryl, C₁-C₂₀ alkyl substituted with one or more groups selected from OH, —O—C₁-C₂₀ alkyl, C₁-C₂₀ alkyl-C₆-C₂₀ aryl, C₁-C₂₀ dialkyl-C₆-C₂₀ aryl, C₁-C₂₀ alkylheterocyclyl, C₁-C₂₀ dialkylheterocyclyl, C₁-C₂₀ alkylheteroaryl, C₁-C₂₀ dialkylheteroaryl, and the like. In some aspects, the linker can comprise a polyalkyleneoxy group. Non-limiting examples of suitable alkyl substituted with one or more heteroatoms selected from O and S include (poly)alkyleneoxy (e.g., (poly)ethyleneoxy and (poly)propyleneoxy, —(NHCH₂CH₂NH)ₓ— wherein x is 1 to about 20, and the like. In certain aspects, the linker is —C(═O)CH₂(OCH₂CH₂)ₘ—NH— or —C(═O)CH₂(OCH₂CH₂)ₘ—NH—C(═O)CH₂(OCH₂CH₂)ₙ—NH—, wherein m and n are independently an integer of from 1 to about 10. A non-limiting example of suitable polyethyleneoxy groups include —(CH₂CH₂O)ₚO— wherein p is an integer of 1 to about 20. Other useful linker moieties include both natural and non-natural amino acids, oligopeptides, e.g., linear or cyclic oligopeptides, and nucleic acids.

In any of the above aspects, the carboxy terminus is —C(═O)—R² wherein R² is OH or a linker, wherein the linker is of length 1 to 120 atoms, and/or has one or some of the elements: C, N, O, S, P, and Si, and/or is in a chain that contains only one or a combination of the following bonds: a single bond, a double bond, a triple bond, an amide bond, an ester bond, a disulfide bond, an imino group, an ether bond, a thioether bond, and a thioester bond. In some aspects, R² can be any suitable linking group for use in solid phase oligonucleotide synthesis, many of which are well known in the art. In some aspects, R² is selected from —C(═O)NH(CH₂CH₂O)ₙCH₂CONH₂, and —NH(CH₂CH₂)ₒNH₂, wherein n and o are independently integers of from 1 to about 10.

In certain aspects, the oligomer comprises a monomer sequence that is complementary to a target nucleotide sequence.

In certain aspects, the target nucleotide comprises RNA.

In certain aspects, the target nucleotide comprises DNA.

The target nucleotide can be any nucleotide, natural or synthetic. According to the well-known principles of Watson-Crick base pairing. When two oligonucleotides comprise naturally occurring nucleobases, purine-pyrimidine base-pairing of AT or GC or UA (in RNA) results in proper duplex structure. Thus, a portion of the inventive oligonucleotide is complementary to a target nucleotide sequence when the aforesaid base pairing occurs. For example, the following sequences are considered to be complementary:
Inventive oligonucleotide and DNA target oligonucleotide:

Inventive oligonucleotide - ATCGATTGAGCTCTAGCG
Target DNA oligonucleotide - TAGCTAACTCGAGATCGC.

Inventive oligonucleotide and RNA target oligonucleotide:

Inventive oligonucleotide - AUCGAUUGAGCUCUAGCG
Target RNA oligonucleotide - UAGCUAACUCGAGAUCGC.

Thus, given a sequence of a target DNA oligonucleotide or portion thereof or a sequence of a target RNA oligonucleotide or portion thereof, the ordinarily skilled artisan can readily design a sequence for an inventive oligonucleotide that is complementary to the sequence of the target oligonucleotide. Alternatively, given the sequence of an inventive oligonucleotide, duplex formation with at least a portion of a target oligonucleotide allows the ordinarily skilled artisan to readily deduce the nucleobase sequence of the portion of the target oligonucleotide. Methods for duplex formation are well known in the art.

In certain preferred aspects, the target nucleotide is selected from miRNA, mRNA, siRNA, ribosomal RNA (rRNA), viral RNA, viral DNA, bacterial RNA, and bacterial DNA. As is well known in the art, microRNA is a small non-coding RNA molecule (containing about 22 nucleotides) found in plants, animals and some viruses, that functions in RNA silencing and post-transcriptional regulation of gene expression. miRNAs function via base-pairing with complementary sequences within messenger RNA (mRNA) molecules. The base-pairing results in silencing of the mRNA molecules by one or more of the following processes: (1) cleavage of the mRNA strand into two pieces, (2) destabilization of the mRNA through shortening of its poly(A) tail, and (3) less efficient translation of the mRNA into proteins by ribosomes. Small interfering RNA (siRNA), also referred to as short interfering RNA or silencing RNA, represents a class of double-stranded RNA non-coding RNA molecules having 20-25 base pairs, which operate within the RNA interference (RNAi) pathway. siRNA interferes with the expression of specific genes with complementary nucleotide sequences by degrading mRNA after transcription, thereby preventing translation.

The inventive oligomer can have a nucleobase sequence designed to complement the known nucleobase sequence of any particular DNA or RNA, for example, any particular microRNA or siRNA, to thereby suppress the biological effects of the DNA or RNA. Desirably, the inventive oligomer possesses a significantly greater water solubility than aegPNA or PNA comprising monomer units of formula (III). When cells are treated with the inventive oligomer, it has been surprisingly found that the inventive oligomer is cell permeable and taken up by cells, whereupon the inventive oligomer has been found to suppress expression of, for example, a microRNA.

In another aspect, the invention provides a compound formula (IV):

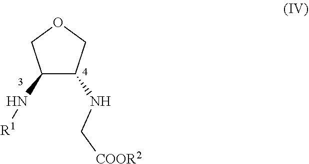

wherein R¹ is a nitrogen protecting group and wherein R² is selected from H, C₁-C₁₀ alkyl, C₆-C₁₀ aryl, and C₁-C₁₀ alkyl-C₆-C₁₀ aryl, wherein the configuration at positions 3 and 4 of the tetrahydrofuran ring is (3R,4R) or (3S,4S).

In certain aspects, R¹ is selected from tert-butoxycarbonyl (Boc), benzhydryloxycarbonyl (Bhoc), and benzyloxycarbonyl.

In certain of these aspects, R¹ is H or C₁-C₁₀ alkyl.

In a further aspect, the invention provides a compound of formula (V):

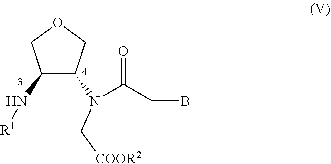

wherein R¹ is a nitrogen protecting group, wherein R² is selected from H, C₁-C₁₀ alkyl, C₆-C₁₀ aryl, and C₁-C₁₀ alkyl-C₆-C₁₀ aryl, and wherein B is an optionally protected nucleobase, wherein the configuration at positions 3 and 4 of the tetrahydrofuran ring is (3R,4R) or (3S,4S).

In certain aspects, B is selected from adenine, 4-(benzhydryloxycarbonyl)adenine, guanosine, 2-(benzhydryloxycarbonyl)guanosine, cytosine, 2-(benzhydryloxycarbonyl)cytosine, thymine, and uracil.

In certain preferred aspects, B is selected from 4-(benzhydryloxycarbonyl)adenine, 2-(benzhydryloxycarbonyl)guanosine, 4-(benzhydryloxycarbonyl)cytosine, thymine, and uracil

In certain of these aspects, R² is H or C₁-C₁₀ alkyl.

In certain particular aspects, the compound is an R,R-enantiomer of the formula:

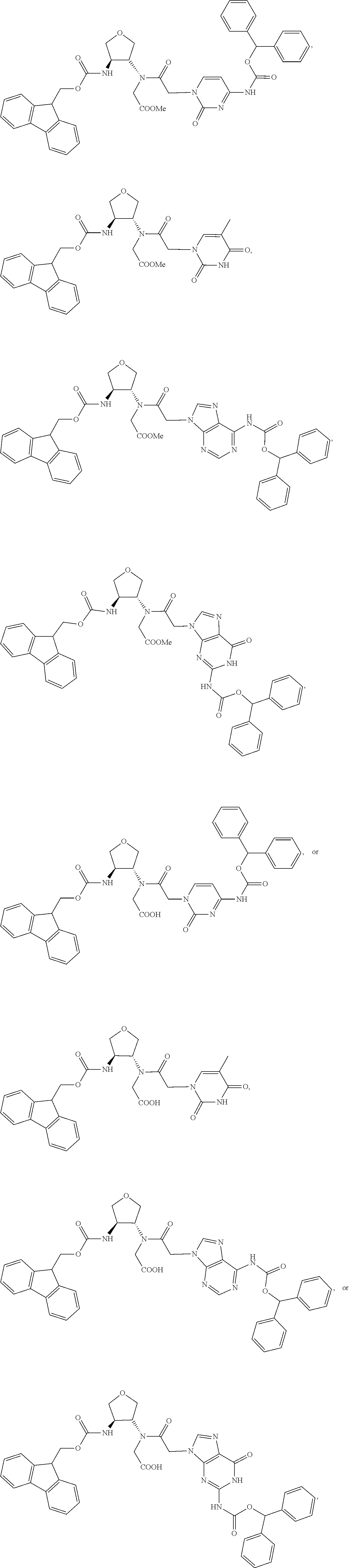

or an S,S-enantiomer of the formula:

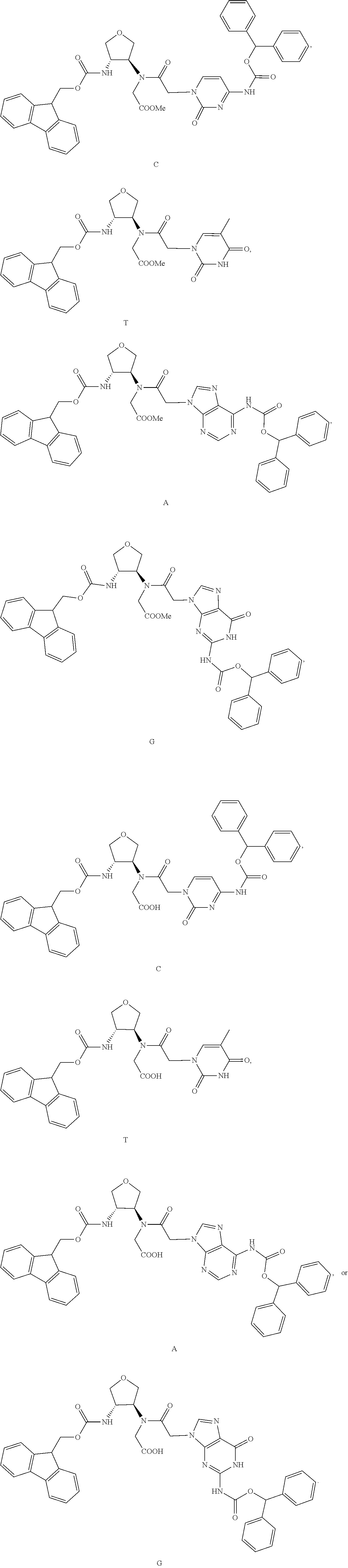

Chemistry

A route to the R,R monomer of formula (I) is shown in Scheme 1.

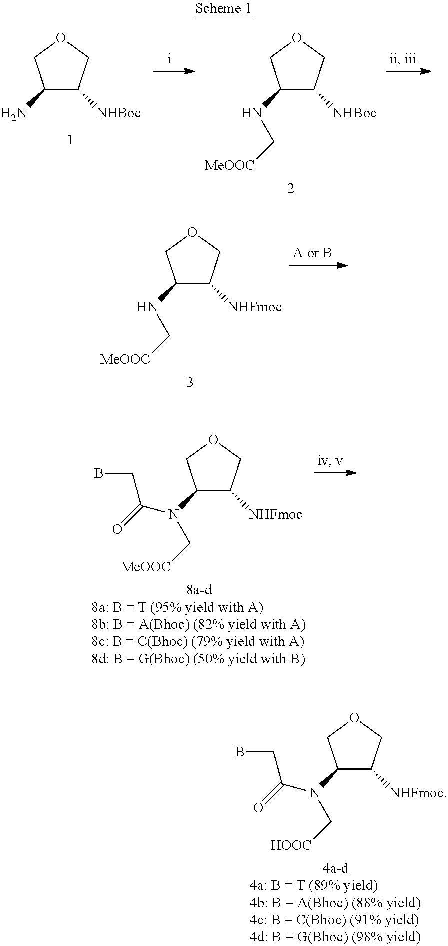

Conditions: i) BrCH₂COOMe (1.0 equiv), Et₃N (1.0 equiv), DMF, 0° C. to rt, slow addition overnight; ii) TFA/DCM (1:2), rt, 5 h; iii) Fmoc-OSu (1.0 equiv), NaHCO₃ (4.0 equiv), dioxane/H₂O, 0° C. to rt, overnight (72% yield for steps ii and iii); A) B—COOH (1.4 equiv), EDC⋅HCl (2.0 equiv), HOBt (10 mole %), DMF, 0° C. to rt, overnight); B) B—COOH (1.4 equiv), HATU (2.5 equiv), DIPEA (4.5 equiv), DMF, rt, 5 h.

tert-butyl ((3R,4R)-4-aminotetrahydrofuran-3-yl)carbamate 1 can be prepared as previously described (A. Luna et al., Org. Lett. 4: 3627 (2002)). Compound 1 can be alkylated with a halo ester such as methyl bromoacetate in the presence of a base such as trimethylamine in a solvent such as DMF to provide compound 2. Compound 2 can be deprotected at the 3-amino group with an acid such as TFA in a solvent such as DCM and then protected at the 3-amino group with another protecting group such as fluorenylmethoxycarbonyl (Fmoc) by reaction with a reagent such as fluorenylmethyloxycarbonyl chloride (Fmoc-Cl) or 9-fluorenylmethylsuccinimidyl carbonate (Fmoc-OSu) in the presence of a base such as NaHCO₃ in a solvent or mixture of solvents such as dioxane/H₂O to provide Fmoc-protected compound 3 in 72% yield, after purification, over three steps. The coupling of nucleobase carboxylic acids and the chiral amine 3 in the presence of a coupling reagent such as N-ethyl-N-(3-dimethylaminopropyl)carbodiimide hydrochloride (EDC) and hydroxybenzotriazole (HOBt) afforded the fully protected thyclotide monomer 4. As a result, the three methyl esters with thymine, adenine, and cytosine 8a-8c could be obtained smoothly. When this coupling step was applied for the preparation of methyl ester 8d with guanine, it could not go to completion and afforded a low yield of 8d. After screening a variety of well-established coupling conditions for amide formation, (1-[bis(dimethylamino)methylene]-1H-1,2,3-triazolo[4,5-b]pyridinum 3-oxide hexafluorophosphare (HATU) and N,N-diisopropylethylamine (DIPEA) were identified to be the optimal combination to furnish 8d in 50% yield. Finally, the methyl esters 8a-8d were subjected to a one-pot sequential hydrolysis/Fmoc reprotection to successfully generate the desired thyclotide monomers 4, which were used directly in the solid-phase peptide synthesis (SPPS) of thyclotide. This synthetic route has been used to produce 8a-8d in five steps with 35-61% overall yields.

The S,S-enantiomers can be prepared in substantially the same manner as the R,R-enantiomers. For example, a synthetic route to prepare S,S-monomers of formula (I) is shown in Scheme 2.

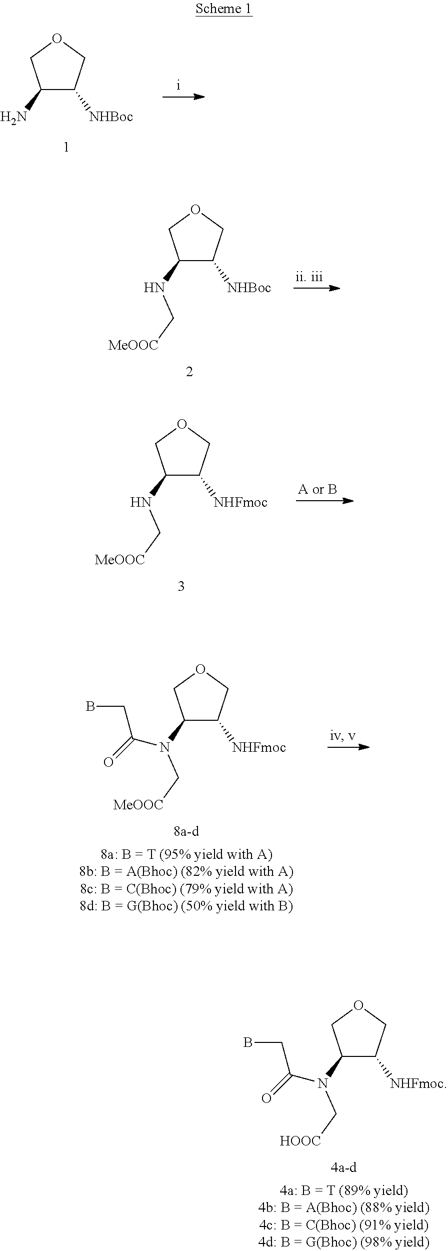

To test the compatibility of the thyclotide monomers with standard Fmoc-based solid-phase synthesis, 4a-4d were incorporated into a 9-residue mixed-base PNA sequence that has been systematically studied. Four different thyclotides 3a-3d, one tcypPNA 2a and one mixed PNA 9 (entry 6 in Table 1) were prepared on 5 μmol scale using Fmoc-based SPPS protocols on an Applied Biosystems 433a automated peptide synthesizer with HATU as the amide-forming reagent (Englund, E. A. et al., Nat. Commun. 3: 614 (2012); (Zhao, C. et al., Nat. Commun. 5: 5079t (2014)). After the completion of oligomerization, the resins were treated with 5% m-cresol in TFA for deprotection and cleavage from the resin. In each PNA, a linker molecule, 2-aminoethoxy-2-ethoxy acetic acid (AEEA), was present at the C-terminal of the first residue and the N-terminal was left unprotected in order to promote aqueous solubility and prevent aggregation. The crude PNAs were readily purified by reversed-phase high-pressure liquid chromatography (RP-HPLC) and characterized by ESI-TOF mass spectrometry (Table 1). Table 1 also includes PNAs (entries 8 and 9) prepared from S,S-enantiomers shown in Scheme 2.

TABLE 1
Mass Characterization Data for PNAs

| entry^{a} | sequence | calcd | obsd^{b} |
|---|---|---|---|
| 1 | GATGTGATA-AEEA (1a) | 1331.0 | 1330.9 |
| 2 | GATGT*GATA-AEEA (3a) | 2702.1 | 2702.7 |
| 3 | GAT*GTGAT*A-AEEA (3b) | 2744.1 | 2744.1 |
| 4 | GAT*GT*GAT*A-AEEA (3c) | 2786.1 | 2786.1 |
| 5 | G*A*T*G*T*G*A*T*A*-AEEA | 1520.1 | 1520.1 |
| | (3d) | | |
| 6 | G*A*T*G#T#G#A*T*A*-AEEA | 1517.1 | 1517.1 |
| | (9) | | |
| 7 | G#A#T#G#T#G#A#T#A#-AEEA | 3021.4 | 3021.2 |
| | (2a) | | |
| 8 | Thiol-dPEG₂₄- | 1663.7 | 1663.7 |
| | A#C#T#T#T#G#A#G#A#C#T#A# | | |
| 9 | Thiol-dPEG₂₄- | 1523.7 | 1523.7 |
| | ACT#TTGAGACT#A | | |

^{a}Cyclopentane stereochemistry is (S,S) and tetrahydrofuran stereochemistry is (R,R), unless indicated otherwise; B* = thf (tetrahydrofuranyl) residue, B# = tcyp (trans-cyclopentyl) residue. AEEA = 2-aminoethoxy-2-ethoxy acetic acid. The data in this table correspond to the singly charged ion [M + H]⁺, doubly charged ion [M + 2H]²⁺, or triply charged ion [M + 3H]³⁺ of PNAs.
^{b}PNAs were characterized using a Waters Xevo-G2 XS qTOFTM instrument. All PNA oligomers gave molecular ions consistent with the final product.

Salts: The phrase “pharmaceutically acceptable salt” is intended to include non-toxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, non-aqueous media such as ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445, and Journal of Pharmaceutical Science, 66, 2-19 (1977).

Suitable bases for forming salts include inorganic bases such as alkali and alkaline earth metal bases, such as those containing metallic cations such as sodium, potassium, magnesium, calcium and the like. Non-limiting examples of suitable bases include sodium hydroxide, potassium hydroxide, sodium carbonate, and potassium carbonate. Suitable acids for forming salts include inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, maleic acid, tartaric acid, fatty acids, long chain fatty acids, and the like. Preferred pharmaceutically acceptable salts of inventive compounds having an acidic moiety include sodium and potassium salts. Preferred pharmaceutically acceptable salts of inventive compounds having a basic moiety (such as a dimethylaminoalkyl group) include hydrochloride and hydrobromide salts. The compounds of the present invention containing an acidic or basic moiety are useful in the form of the free base or acid or in the form of a pharmaceutically acceptable salt thereof.

It should be recognized that the particular counterion forming a part of any salt of this invention is usually not of a critical nature, so long as the salt as a whole is pharmacologically acceptable and as long as the counterion does not contribute undesired qualities to the salt as a whole.

It is further understood that the above compounds and salts may form solvates, or exist in a substantially uncomplexed form, such as the anhydrous form. As used herein, the term “solvate” refers to a molecular complex wherein the solvent molecule, such as the crystallizing solvent, is incorporated into the crystal lattice. When the solvent incorporated in the solvate is water, the molecular complex is called a hydrate. Pharmaceutically acceptable solvates include hydrates, alcoholates such as ethanolates, acetonitrilates and the like. These compounds can also exist in polymorphic forms.

The present invention is further directed to a pharmaceutical composition comprising a pharmaceutically acceptable carrier and at least one compound or salt described herein.

It is preferred that the pharmaceutically acceptable carrier be one that is chemically inert to the active compounds and one that has no detrimental side effects or toxicity under the conditions of use.

The choice of carrier will be determined in part by the particular compound of the present invention chosen, as well as by the particular method used to administer the composition. Accordingly, there is a wide variety of suitable formulations of the pharmaceutical composition of the present invention. The following formulations for oral, aerosol, nasal, pulmonary, parenteral, subcutaneous, intravenous, intramuscular, intraperitoneal, intrathecal, intratumoral, topical, rectal, and vaginal administration are merely exemplary and are in no way limiting.

The pharmaceutical composition can be administered parenterally, such as intravenously, subcutaneously, intradermally, or intramuscularly. Thus, the invention provides compositions for parenteral administration that comprise a solution or suspension of the inventive compound or salt dissolved or suspended in an acceptable carrier suitable for parenteral administration, including aqueous and non-aqueous isotonic sterile injection solutions.

Overall, the requirements for effective pharmaceutical carriers for parenteral compositions are well known to those of ordinary skill in the art. Such solutions can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound or salt of the present invention may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

The parenteral formulations can contain preservatives and buffers. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Topical formulations, including those that are useful for transdermal drug release, are well-known to those of skill in the art and are suitable in the context of the invention for application to skin. Topically applied compositions are generally in the form of liquids, creams, pastes, lotions and gels. Topical administration includes application to the oral mucosa, which includes the oral cavity, oral epithelium, palate, gingival, and the nasal mucosa. In some aspects, the composition contains at least one active component and a suitable vehicle or carrier. It may also contain other components, such as an anti-irritant. The carrier can be a liquid, solid or semi-solid. In aspects, the composition is an aqueous solution. Alternatively, the composition can be a dispersion, emulsion, gel, lotion or cream vehicle for the various components. In one aspect, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In aspects of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

Formulations suitable for oral administration can consist of (a) liquid solutions, such as a therapeutically effective amount of the inventive compound dissolved in diluents, such as water, saline, or orange juice, (b) capsules, sachets, tablets, lozenges, and troches, each containing a predetermined amount of the active ingredient, as solids or granules, (c) powders, (d) suspensions in an appropriate liquid, and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The compound or salt of the present invention, alone or in combination with other suitable components, can be made into aerosol formulations to be administered via inhalation. The compounds are preferably supplied in finely divided form along with a surfactant and propellant. Typical percentages of active compound are 0.01%-20% by weight, preferably 1%-10%. The surfactant must, of course, be nontoxic, and preferably soluble in the propellant. Representative of such surfactants are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25%-5%. The balance of the composition is ordinarily propellant. A carrier can also be included as desired, such as lecithin for intranasal delivery. These aerosol formulations can be placed into acceptable pressurized propellants, such as dichlorodifluoromethane, propane, nitrogen, and the like. They also may be formulated as pharmaceuticals for non-pressured preparations, such as in a nebulizer or an atomizer. Such spray formulations may be used to spray mucosa.

Additionally, the compound or salt of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

It will be appreciated by one of ordinary skill in the art that, in addition to the above described pharmaceutical compositions, the compound or salt of the present invention may be formulated as inclusion complexes, such as cyclodextrin inclusion complexes, or liposomes. Liposomes serve to target the compounds to a particular tissue, such as lymphoid tissue or cancerous hepatic cells. Liposomes can also be used to increase the half-life of the inventive compound. Liposomes useful in the present invention include emulsions, foams, micelles, insoluble monolayers, liquid crystals, phospholipid dispersions, lamellar layers and the like. In these preparations, the active agent to be delivered is incorporated as part of a liposome, alone or in conjunction with a suitable chemotherapeutic agent. Thus, liposomes filled with a desired inventive compound or salt thereof, can be directed to the site of a specific tissue type, hepatic cells, for example, where the liposomes then deliver the selected compositions. Liposomes for use in the invention are formed from standard vesicle-forming lipids, which generally include neutral and negatively charged phospholipids and a sterol, such as cholesterol. The selection of lipids is generally guided by consideration of, for example, liposome size and stability of the liposomes in the blood stream. A variety of methods are available for preparing liposomes, as described in, for example, Szoka et al., Ann. Rev. Biophys. Bioeng., 9, 467 (1980), and U.S. Pat. Nos. 4,235,871, 4,501,728, 4,837,028, and 5,019,369. For targeting to the cells of a particular tissue type, a ligand to be incorporated into the liposome can include, for example, antibodies or fragments thereof specific for cell surface determinants of the targeted tissue type. A liposome suspension containing a compound or salt of the present invention may be administered intravenously, locally, topically, etc. in a dose that varies according to the mode of administration, the agent being delivered, and the stage of disease being treated. Encapsulation formulations including liposome formulations are well suited for use in oral administration of the inventive oligomer, optionally further comprising permeation enhancers such as sodium caprate (see, e.g., van Putten, M. et al, Mol Ther Nucleic Acids, November; 3(11): e211 (2014).

The compounds or salts thereof can be used in any suitable dose. Suitable doses and dosage regimens can be determined by conventional range finding techniques. Generally, treatment is initiated with smaller dosages, which are less than the optimum dose. Thereafter, the dosage is increased by small increments until optimum effect under the circumstances is reached. For convenience, the total daily dosage may be divided and administered in portions during the day if desired. In proper doses and with suitable administration of certain compounds, the present invention provides for a wide range of responses. Typically, the dosages range from about 0.001 to about 1000 mg/kg body weight of the animal being treated/day. For example, in aspects, the compounds or salts may be administered from about 100 mg/kg to about 300 mg/kg, from about 120 mg/kg to about 280 mg/kg, from about 140 mg/kg to about 260 mg/kg, from about 150 mg/kg to about 250 mg/kg, from about 160 mg/kg to about 240 mg/kg, of subject body weight per day, one or more times a day, to obtain the desired therapeutic effect.

The following examples further illustrate the invention but, of course, should not be construed as in any way limiting its scope.

Unless otherwise stated, all reactions were performed under a nitrogen atmosphere using flame-dried glassware and all reagents were reagent grade quality and used as received from Aldrich. (1R,2R)-trans-N-Boc-4-oxa-1,2-Cyclopentanediamine (>95% purity, >99% e.e.) was purchased from EntreChem SL (Spain) and used as received. All nucleobase acetic acids were purchased from PolyOrg, Inc. (MA, USA). Anhydrous 1,4-Dioxane (99.8%), N,N-Dimethylformamide (99.8%) and Dichloromethane (>99.8%, contains 40-150 ppm amylene as stabilizer) were purchased from Sigma-Aldrich and used as received. Thin layer chromatography (TLC) was performed on SiliCycle Silica Gel 60 F254 plates and was visualized with UV light and KMnO₄ stain. All NMR spectra were recorded on either a Bruker Avance 500 MHz or 400 MHz spectrometer at STP. All deuterated solvents were used as received from Cambridge Isotope Laboratories, Inc. The residual solvent protons (¹H) or the solvent carbons (¹³C) were used as internal standards. ¹H NMR data are presented as follows: chemical shift in ppm (δ) downfield from tetramethylsilane (multiplicity, coupling constant, integration). The following abbreviations are used in reporting NMR data: s, singlet; d, doublet; t, triplet; q, quartet; quint, quintet; dd, doublet of doublets; m, multiplet. High-resolution mass spectrometry (HRMS) data were obtained using a Waters Xevo-G2 XS qTOF™ instrument.

### Example 1

This example demonstrates syntheses of monomers 4 (see Scheme 1) in accordance with an aspect of the invention.

Step 1: To a solution of tert-butyl ((3R,4R)-4-aminotetrahydrofuran-3-yl)carbamate 5 (4.04 g, 20.0 mmol) and triethylamine (3.79 mL, 20.0 mmol) in DMF (10 mL) at 0° C. was slowly added a solution of methyl 2-bromoacetate (3.06 g, 20.0 mmol) in DMF (10 mL) dropwise. The resulting solution was allowed to warm to room temperature and stirred at room temperature overnight. The resulting mixture was extracted between EtOAc (3×30 mL) and a saturated aqueous NaHCO₃ solution (30 mL). The combined organic layers were washed with brine, dried over Na₂SO₄, filtered, and evaporated under reduced pressure to provide the crude product 6 as a pale-yellow oil, which was used in next step without any further purification.

Steps 2 and 3: To a solution of crude 6 (from step 1) in CH₂Cl₂ (20 mL) at room temperature was added trifluoroacetic acid (10 mL). The resulting solution was stirred at room temperature for 5 hours. Upon evaporation of solvent, the crude amine TFA salt was obtained as a yellow oil. To a solution of this freshly prepared crude amine TFA salt and NaHCO₃ (6.88 g, 80.0 mmol) in dioxane/H₂O (20 mL/20 mL) at 0° C. was slowly added a solution of Fmoc-OSu (6.74 g, 20.0 mmol) in dioxane (20 mL). The resulting mixture was allowed to warm to room temperature and stirred at room temperature overnight. The resulting mixture was extracted between EtOAc (3×50 mL) and H₂O (30 mL). The combined organic layers were washed with brine, dried over Na₂SO₄, filtered, and evaporated under reduced pressure. The residue was purified by silica gel chromatography (EtOAc:hexanes=1:1 to 4:1) to give the desired compound 7 as a white solid (5.72 g, 72% yield over 3 steps). ¹H NMR (400 MHz, CDCl₃) (7.76 (d, J=7.5 Hz, 2H), 7.57 (d, J=7.4 Hz, 2H), 7.39 (t, J=7.4 Hz, 2H), 7.31 (t, J=7.4 Hz, 2H), 5.14 (d, J=6.6 Hz, 1H), 4.43 (d, J=5.8 Hz, 2H), 4.19 (t, J=6.3 Hz, 1H), 4.08-3.94 (m, 3H), 3.72 (s, 3H), 3.63 (d, J=9.2 Hz, 1H), 3.54 (s, 2H), 3.48 (dd, J=9.1, 3.0 Hz, 1H), 3.21-3.16 (m, 1H), 2.00-1.70 (br s, 1H); ¹³C NMR (100 MHz, CDCl₃) (173.2, 156.2, 144.2, 141.7, 128.1, 127.4, 125.3, 120.4, 73.4, 72.1, 66.9, 65.6, 57.8, 52.3, 49.2, 47.6; HRMS (ESI) for C₂₂H₂₅N₂O₅: calcd. 397.1763; found 397.1758.

Step 4 (Method A): To a solution of 7 (1.19 g, 3.0 mmol), C(Bhoc)CH₂COOH (1.59 g, 4.2 mmol) and HOBt (40 mg, 0.3 mmol) in DMF (10 mL) at 0° C. was added EDC (1.15 g, 6.0 mmol) in one portion. The resulting mixture was allowed to warm to room temperature and stirred at room temperature overnight. The resulting mixture was extracted between EtOAc (3×50 mL) and H₂O (50 mL). The combined organic layers were washed with brine, dried over Na₂SO₄, filtered, and evaporated under reduced pressure. The residue was purified by silica gel chromatography (CH₂Cl₂:MeOH=60:1 to 20:1) to give the desired compound 8c as a white solid (1.79 g, 79% yield).

Compounds 8a and 8b were prepared following the same procedure as that described for the preparation of 8c.

8a: ¹H NMR (500 MHz, CDCl₃): Major rotamer: (9.64 (s, 1H), 7.75-7.65 (m, 2H), 7.60-7.50 (m, 2H), 7.40-7.32 (m, 2H), 7.29-7.22 (m, 2H), 6.97 (s, 1H), 5.94 (s, 1H), 4.84-4.30 (m, 5H), 4.27-3.85 (m, 7H), 3.68 (s, 3H), 3.62-3.55 (m, 1H), 1.85 (s, 3H); Minor rotamer: (9.46 (s, 1H), 7.75-7.65 (m, 2H), 7.60-7.50 (m, 2H), 7.40-7.32 (m, 2H), 7.29-7.22 (m, 2H), 6.97 (s, 1H), 5.57 (s, 1H), 4.84-4.30 (m, 5H), 4.27-3.85 (m, 7H), 3.78 (s, 3H), 3.55-3.48 (m, 1H), 1.91 (s, 3H); ¹³C NMR (125 MHz, CDCl₃): Major rotamer: δ 169.8, 167.5, 164.4, 156.1, 151.5, 143.7, 141.4, 141.2, 127.8, 127.1, 124.9, 120.1, 110.9, 71.1, 69.4, 66.6, 62.6, 58.2, 52.5, 48.1, 47.2, 44.8, 12.4; Minor rotamer: (170.0, 168.1, 164.4, 156.1, 151.2, 143.5, 141.3, 140.9, 127.8, 127.1, 125.1, 120.1, 111.0, 72.4, 68.5, 66.9, 63.1, 56.2, 53.1, 48.3, 47.1, 46.8, 12.4; HRMS (ESI) for C₂₉H₃₁N₄O₈: calcd. 563.2142; found 563.2148.

8b: ¹H NMR (500 MHz, CDCl₃): Major rotamer: (8.66 (s, 1H), 8.06 (s, 1H), 7.70-7.60 (m, 2H), 7.51-7.44 (m, 2H), 7.40-7.16 (m, 15H), 6.95 (s, 1H), 5.86 (s, 1H), 5.50-5.40 (m, 1H), 4.97-4.89 (m, 1H), 4.53-3.85 (m, 9H), 3.80-3.45 (m, 5H); Minor rotamer: δ 8.66 (s, 1H), 8.06 (s, 1H), 7.70-7.60 (m, 2H), 7.51-7.44 (m, 2H), 7.40-7.16 (m, 15H), 6.95 (s, 1H), 5.50 (s, 1H), 5.20-5.10 (m, 1H), 4.62-4.56 (m, 1H), 4.53-3.85 (m, 9H), 3.80-3.45 (m, 5H); ¹³C NMR (125 MHz, CDCl₃): Major rotamer: (169.7, 166.6, 156.2, 152.7, 151.5, 150.3, 149.1, 144.2, 143.6, 141.3, 139.6, 128.6, 128.1, 127.8, 127.7, 127.3, 127.0, 124.8, 120.0, 78.8, 71.2, 69.2, 66.5, 63.0, 58.4, 52.5, 47.2, 45.0, 44.3; Minor rotamer: (167.7, 167.0, 156.2, 152.8, 151.4, 150.3, 149.2, 144.0, 143.4, 141.4, 141.3, 128.6, 128.1, 127.8, 127.7, 127.3, 127.0, 125.0, 120.9, 78.8, 72.3, 68.4, 66.8, 63.6, 56.1, 53.2, 47.1, 45.0, 44.3; HRMS (ESI) for C₄₃H₄₀N₇O₈: calcd. 782.2938; found 782.2937.

8c: ¹H NMR (500 MHz, CDCl₃): Major rotamer: (8.28 (br s, 1H), 7.82-7.65 (m, 2H), 7.60-7.07 (m, 18H), 6.80-6.73 (m, 1H), 5.37-5.20 (m, 1H), 5.00-3.50 (m, 16H); Minor rotamer: (8.28 (br s, 1H), 7.82-7.65 (m, 2H), 7.60-7.07 (m, 18H), 6.70-6.63 (m, 1H), 5.73-5.57 (m, 1H), 5.00-3.50 (m, 16H); ¹³C NMR (125 MHz, CDCl₃): Major rotamer: δ 169.7, 167.3, 163.1, 156.3, 155.7, 151.7, 143.74, 143.66, 141.3, 139.4, 128.6, 128.2, 127.8, 127.1, 126.9, 125.1, 120.0, 95.3, 79.0, 71.4, 69.4, 66.8, 62.5, 57.5, 52.4, 50.0, 47.1, 44.8; Minor rotamer: (169.4, 167.1, 163.1, 157.4, 156.2, 150.1, 143.74, 143.66, 141.3, 139.4, 128.6, 128.2, 128.0, 127.3, 126.9, 124.9, 120.0, 95.3, 78.8, 72.4, 69.9, 67.2, 61.0, 57.1, 53.0, 50.2, 46.7, 44.8; HRMS (ESI) for C₄₂H₄₀N₅O₉: calcd. 758.2826; found 758.2833.

Step 4 (Method B): A suspension of 7 (1.00 g, 2.52 mmol), G(Bhoc)CH₂COOH (1.48 g, 3.53 mmol), and 1-[bis(dimethylamino)methylene]-1H-1,2,3-triazolo[4,5-b]pyridinium 3-oxide hexafluorophosphate (HATU) (2.42 g, 6.36 mmol) in toluene (10 mL) was evaporated under reduced pressure to remove water. The resulting mixture was dissolved in DMF (freshly dried over 4 Å molecular sieves, 5 mL) and N,N-diisopropylethylamine (2.0 ml, 11.30 mmol) was added in one portion. The resulting solution was stirred at room temperature for 5 hours. The resulting mixture was extracted between EtOAc (3×50 mL) and H₂O (50 mL). The combined organic layers were washed with brine, dried over Na₂SO₄, filtered, and evaporated under reduced pressure. The residue was purified by Biotage© Selekt column chromatography system using Biotage© SNAP KP-Sil 100 g silica gel column (EtOAc:hexanes=0:100 to 100:0, then EtOAc/MeOH=100:0 to 3:1) to give the desired compound 8d as a white solid (1.25 g, 62% yield). ¹H NMR (500 MHz, DMSO-d₆ with one drop of CDCl₃): Major rotamer: δ 11.70 (s, 1H), 11.27 (s, 1H), 7.90-7.60 (m, 6H), 7.50-7.20 (m, 14H), 6.85 (s, 1H), 5.23 (s, 2H), 4.60-4.17 (m, 5H), 4.10-3.74 (m, 5H), 3.72-3.46 (m, 4H); Minor rotamer: δ 11.76 (s, 1H), 11.27 (s, 1H), 7.90-7.60 (m, 6H), 7.50-7.20 (m, 14H), 6.85 (s, 1H), 4.98 (s, 2H), 4.60-4.17 (m, 5H), 4.10-3.74 (m, 5H), 3.72-3.46 (m, 4H); ¹³C NMR (125 MHz, DMSO-d₆ with one drop of CDCl₃): Major rotamer: δ 170.0, 167.2, 156.6, 155.5, 154.3, 150.0, 147.6, 144.2, 141.3, 140.6, 140.2, 128.9, 128.4, 128.0, 127.4, 127.0, 125.5, 120.4, 119.7, 78.6, 71.9, 69.1, 66.0, 62.3, 57.1, 52.3, 47.3, 45.0, 44.8; Minor rotamer: δ 170.6, 167.8, 156.3, 155.5, 154.3, 150.0, 147.6, 144.2, 141.3, 140.7, 140.2, 128.9, 128.4, 128.0, 127.4, 127.0, 125.4, 120.4, 119.6, 78.6, 72.5, 68.8, 66.0, 62.8, 55.2, 52.7, 47.2, 44.9, 44.8, HRMS (ESI) for C₄₃H₄₀N₇O₉: calcd. 798.2888; found 798.2883.

Step 5: To a solution of 8c (1.42 g, 1.87 mmol) in dioxane/H₂O (20 mL/10 mL) at 0° C. was added LiOH—H₂O (196 mg, 4.68 mmol). The resulting solution was allowed to warm to room temperature and stirred at room temperature for 30 minutes. Then 20% citric acid was added to adjust the reaction mixture to pH 7 and NaHCO₃ was subsequently added to adjust the reaction mixture to pH 8. The resulting mixture was cooled to 0° C. and Fmoc-OSu (630 mg, 1.87 mmol) was added. The resulting solution was allowed to warm to room temperature and stirred at room temperature overnight. The reaction mixture was washed with Et₂O (3×30 mL). The aqueous layer was acidified with 20% citric acid to pH 3 and then extracted with EtOAc (3×50 mL). The combined organic layers were washed with brine, dried over Na₂SO₄, filtered, and evaporated under reduced pressure. The residue was saturated with Et₂O (30 mL) to provide a precipitate which was filtered and washed with Et₂O (3×20 mL) to give the desired compound 4c as a white solid (1.26 g, 91% yield).

Compounds 4a, 4b, and 4d were prepared following the same procedure as that described for the preparation of 4c.

4a: ¹H NMR (500 MHz, DMSO-d₆): Major rotamer: (12.66 (br s, 1H), 11.33 (s, 1H), 7.95-7.85 (m, 2H), 7.75-7.65 (m, 2H), 7.50-7.31 (m, 5H), 7.20 (s, 1H), 4.83-4.70 (m, 1H), 4.60-4.10 (m, 6H), 4.09-3.77 (m, 4H), 3.50-3.45 (m, 1H), 3.43-3.35 (m, 1H), 1.72 (s, 3H); Minor rotamer: (12.66 (br s, 1H), 11.33 (s, 1H), 7.95-7.85 (m, 2H), 7.75-7.65 (m, 2H), 7.50-7.31 (m, 5H), 7.30 (s, 1H), 4.83-4.70 (m, 1H), 4.60-4.10 (m, 6H), 4.09-3.77 (m, 4H), 3.75-3.67 (m, 1H), 3.43-3.35 (m, 1H), 1.75 (s, 3H); ¹³C NMR (125 MHz, DMSO-d₆): Major rotamer: δ 171.1, 167.8, 164.9, 156.5, 151.5, 144.3, 142.3, 141.2, 128.2, 127.6, 125.6, 120.6, 108.8, 71.6, 68.8, 66.0, 62.0, 56.4, 48.5, 47.2, 45.0, 12.4; Minor rotamer: δ 171.6, 168.4, 164.9, 156.3, 151.5, 144.4, 142.6, 141.2, 128.2, 127.6, 125.6, 120.6, 108.7, 72.8, 68.7, 65.9, 62.4, 55.4, 48.8, 47.2, 45.0, 12.4; HRMS (ESI) for C₂₈H₂₉N₄O₈: calcd. 549.1985; found 549.1980.

4b: ¹H NMR (500 MHz, DMSO-d₆): Major rotamer: (12.68 (br s, 1H), 10.96 (s, 1H), 8.53 (s, 1H), 8.31 (s, 1H), 7.96-7.65 (m, 5H), 7.60-7.50 (m, 4H), 7.45-7.25 (m, 10H), 6.84 (s, 1H), 5.54-5.40 (m, 1H), 5.26-5.15 (m, 1H), 4.64-4.42 (m, 2H), 4.37-3.80 (m, 7H), 3.75-3.35 (m, 2H); Minor rotamer: (12.68 (br s, 1H), 10.96 (s, 1H), 8.60 (s, 1H), 8.37 (s, 1H), 7.96-7.65 (m, 5H), 7.60-7.50 (m, 4H), 7.45-7.25 (m, 10H), 6.84 (s, 1H), 5.54-5.40 (m, 1H), 5.26-5.15 (m, 1H), 4.64-4.42 (m, 2H), 4.37-3.80 (m, 7H), 3.75-3.35 (m, 2H); ¹³C NMR (125 MHz, DMSO-d₆): Major rotamer: (171.0, 167.1, 156.6, 152.8, 152.0, 151.6, 149.8, 145.6, 144.4, 144.2, 141.4, 129.0, 128.2, 128.1, 127.5, 127.0, 125.6, 123.1, 120.6, 77.7, 71.6, 68.8, 66.0, 62.1, 56.4, 47.2, 45.1, 44.8; Minor rotamer: (171.7, 167.8, 156.3, 152.8, 152.0, 151.6, 149.8, 145.8, 144.4, 144.2, 141.2, 129.8, 128.2, 128.1, 127.6, 127.0, 125.6, 123.1, 120.6, 77.7, 72.7, 68.7, 65.9, 62.3, 55.3, 47.2, 45.1, 44.8; HRMS (ESI) for C₄₂H₃₈N₇O₈: calcd. 768.2782; found 768.2789.

4c: ¹H NMR (500 MHz, DMSO-d₆): Major rotamer: (12.67 (br s, 1H), 11.04 (br s, 1H), 7.92-7.65 (m, 6H), 7.50-7.25 (m, 14H), 6.97-6.93 (m, 1H), 6.82 (s, 1H), 4.95 (s, 1H), 4.67-4.20 (m, 6H), 4.17-3.80 (m, 4H), 3.52-3.45 (m, 1H), 3.42-3.36 (m, 1H); Minor rotamer: (12.67 (br s, 1H), 11.04 (br s, 1H), 7.92-7.65 (m, 6H), 7.50-7.25 (m, 14H), 6.97-6.93 (m, 1H), 6.82 (s, 1H), 4.95 (s, 1H), 4.67-4.20 (m, 6H), 4.17-3.80 (m, 4H), 3.75-3.67 (m, 1H), 3.42-3.36 (m, 1H); ¹³C NMR (125 MHz, DMSO-d₆): Major rotamer: (171.0, 167.7, 163.6, 156.5, 155.4, 151.3, 144.4, 144.3, 141.2, 140.9, 129.1, 128.4, 128.1, 127.6, 126.9, 125.6, 120.6, 94.4, 77.9, 71.8, 68.8, 66.0, 61.8, 56.4, 50.3, 47.2, 45.0; Minor rotamer: (171.6, 168.3, 163.6, 156.3, 152.9, 151.5, 144.4, 144.3, 141.2, 140.9, 129.1, 128.4, 128.1, 127.6, 126.9, 125.6, 120.6, 94.3, 77.9, 72.8, 68.8, 65.9, 62.3, 55.5, 50.6, 47.2, 45.0; HRMS (ESI) for C₄₁H₃₇N₅O₉Na: calcd. 766.2489; found 766.2489.

4d: ¹H NMR (500 MHz, DMSO-d₆): Major rotamer: (13.27 (br s, 1H), 12.71 (br s, 1H), 11.72 (s, 1H), 11.28 (s, 1H), 7.92-7.67 (m, 5H), 7.50-7.25 (m, 14H), 6.88 (s, 1H), 5.30-5.17 (m, 1H), 4.98 (s, 1H), 4.60-4.14 (m, 6H), 4.12-3.80 (m, 3H), 3.55-3.50 (m, 1H), 3.42-3.37 (m, 1H); Minor rotamer: 13.27 (br s, 1H), 12.71 (br s, 1H), 11.78 (s, 1H), 11.27 (s, 1H), 7.92-7.67 (m, 5H), 7.50-7.25 (m, 14H), 6.88 (s, 1H), 5.30-5.17 (m, 1H), 4.98 (s, 1H), 4.60-4.14 (m, 6H), 4.12-3.80 (m, 3H), 3.75-3.68 (m, 1H), 3.42-3.37 (m, 1H); ¹³C NMR (125 MHz, DMSO-d₆): Major rotamer: (171.1, 167.1, 156.6, 155.6, 154.3, 150.0, 147.5, 144.3, 141.3, 140.8, 140.6, 129.1, 128.5, 128.1, 127.5, 126.9, 125.6, 120.6, 119.9, 78.5, 72.0, 69.0, 66.0, 62.2, 56.9, 47.3, 45.2, 44.8; Minor rotamer: δ 171.7, 167.8, 156.3, 155.6, 154.3, 150.0, 147.5, 144.2, 141.2, 141.0, 140.6, 129.1, 128.5, 128.1, 127.6, 126.9, 125.6, 120.6, 119.8, 78.5, 72.8, 68.8, 66.0, 62.5, 55.4, 47.2, 45.2, 45.0; HRMS (ESI) for C₄₂H₃₈N₇O₉: calcd. 784.2731; found 784.2728.

### Example 2

This example demonstrates the preparation of PNA oligomers in accordance with an aspect of the invention.

Reagents and Materials

All 9-fluorenylmethoxycarbonyl (Fmoc)-PNA monomers were purchased from PolyOrg, Inc. (Leominster, Mass., USA). Acetonitrile, acetic anhydride (Ac₂O), pyridine, thioanisole, 1-[bis(dimethylamino)methylene]-1H-1,2,3-triazolo[4,5-b]pyridinium 3-oxid hexafluorophosphate (HATU), dichloromethane (DCM), N,N-diisopropylethylamine (DIPEA), diethyl ether (Et₂O), N,N-dimethylformamide (DMF), Kaiser test reagents, m-cresol, N-methyl-2-pyrrolidinone (NMP), piperidine, and trifluoroacetic acid (TFA) were purchased from Sigma-Aldrich (St Louis, Mo., USA). High purity water (18 MQ) was generated from a Millipore (Billerica, Mass., USA) Milli-Q water system. Methyl-benzhydrylamine (MBHA) resin was purchased from Advanced Chemtech (Louisville, Ky., USA). Fmoc-8-amino-3,6-dioxaoctanoic acid was purchased from Peptides International, Inc. (Louisville, Ky., USA).

Resin Downloading Protocol

MBHA resin was downloaded from 0.3 to 0.1 mmol g⁻¹ using Fmoc-8-amino-3,6-dioxaoctanoic acid. The MBHA resin (1.0 g) was swelled with DCM for 1 hour in a peptide synthesis vessel. The following solutions were prepared: (a) 0.4 M Fmoc-8-amino-3,6-dioxaoctanoic acid in NMP, (b) 0.2 M HATU in DMF and (c) 0.5 M DIPEA in NMP. Next, 450 μL of solution a, 460 μL of solution c and 1.59 mL NMP were combined and mixed to make solution 1. 550 μL of solution b were diluted with NMP (1.95 mL) to make solution 2. Solutions 1 and 2 were combined and pre-mixed for about 30 seconds before adding to the drained, swelled resin. The resin/coupling mixture was agitated for 1 hour before draining and washing with DMF (4×2 mL), DCM (4×2 mL), 5% DIPEA in DCM (1×2 mL for 30 seconds) and finally DCM (4×2 mL). Any remaining active sites were capped using capping cocktail (Ac₂O:NMP:pyridine=1:25:25) for 20 minutes. The reaction was drained and rinsed with DMF (3×2 mL) and DCM (3×2 mL). The progress of the capping was followed by a qualitative Kaiser test. If the test was positive, the resin was resubmitted to capping. After a negative test for primary amines, the resin was washed with DCM (3×2 mL) and dried under vacuum for 30-60 minutes and then stored in a dessicator.

General Method for PNA Synthesis

PNAs were prepared on 5 μmol scale using Fmoc-solid phase peptide synthesis protocols on an Applied Biosystems 433a automated peptide synthesizer with HATU as the amide-forming reagent. All PNA were synthesized on Fmoc-8-amino-3,6-dioxaoctanoic acid downloaded MBHA resin.

Cleavage and Recovery of Crude PNA from Resin

The resin, in a peptide synthesis vessel, was first washed with 20% piperidine in DMF (2×5 mL for 5 min) to deprotect the Fmoc group. The progress of the deprotection was followed by a qualitative Kaiser test. If the test was negative, the resin was resubmitted to additional deprotection. After a positive test for primary amines, the resin was drained and treated with cleavage cocktail (2 mL, 5% m-cresol in TFA) for 1 hour. The cleavage mixture was collected in a glass vial using N₂ pressure to drain the vessel. The resin was resubmitted to fresh cleavage cocktail and cleaved for 1 hour, and was drained into the first cleavage fraction. The volatiles were removed by flowing dry N₂ over the solution to produce a yellow-brown oil. Approximately 10 mL of Et₂O was added to the cleavage oil to create a suspended white precipitate. The suspension was partitioned into five 2 ml microcentrifuge tubes and chilled over dry ice for 10 minutes. The tubes were centrifuged at 12,000 r.p.m. for 40 seconds to produce a white pellet. Et₂O was carefully decanted, leaving the white crude PNA solid. Further washing was performed by adding about 1.6 mL of Et₂O to each tube, mixing to resuspend the precipitate, and chilling on dry ice for 5 minutes. Following centrifugation and decanting, the washes were repeated twice without dry ice. After the final wash, the white precipitate was dried by carefully passing a stream of dry N₂ over the crude PNA.

Purification of Crude PNA and Characterization

Purification was performed on an Agilent (Santa Clara, Calif.) 1260 Series RP-HPLC with automatic fraction collection using ultraviolet detection at 260 nm. Waters (Milford, Mass., USA) XBridge C18 (10×250 mm, 5 μm) columns were used in conjunction with Solvents A and B for purification at 45° C. Waters (Milford, Mass., USA) XBridge C18 (4.6×250 mm, 5 μm) columns were used in conjunction with Solvents A and B for purity checking at room temperature. Solvent A was 0.05% TFA in water and Solvent B consisted of 90% acetonitrile in water. PNA HPLC isolates were characterized using electrospray ionization-mass spectrometry on a Waters/Micromass LCT Premier time-of-flight mass spectrometer. The instrument was operated in W-mode at a nominal resolution of 10,000. The electrospray capillary voltage was 2 kV and the sample cone voltage was 60 V. The desolvation temperature was 275° C. and the desolvation gas was N₂ with a flow of 300 L h⁻¹. Accurate masses were obtained using the internal reference standard method. The sample was introduced into the mass spectrometer via the direct loop injection method. Deconvolution of multiply charged ions was performed with MaxEnt I. All PNA oligomers gave molecular ions consistent with the calculated theoretical product values.

| | | |
|---|---|---|
| Time | A (%) | B (%) |

Purification method for PNAs:

| | | |
|---|---|---|
| 0 | 100 | 0 |
| 2 | 90 | 10 |
| 20 | 60 | 40 |
| 21 | 0 | 100 |
| 25 | 0 | 100 |
| 26 | 100 | 0 |

Purity checking method (3a, 3b, and 3c):

| | | |
|---|---|---|
| 0 | 100 | 0 |
| 2 | 90 | 10 |
| 30 | 80 | 20 |
| 31 | 0 | 100 |
| 35 | 0 | 100 |
| 36 | 100 | 0 |

### Example 3

This example demonstrates effects of tetrahydrofuran substitution in PNAs on the melting temperature for various thyclotides (THF-PNAs) 3a-3d and unmodified aegPNA 1a when bound to complementary DNA, in accordance with an aspect of the invention.

These results showed that incorporation of a single tetrahydrofuran resulted in an increase in the melting temperature (Tₘ) when bound to complimentary DNA (entries 1 and 2, Table 2). Importantly, the extent of stabilization gradually increased with additional tetrahydrofuran units added (entries 3-5). The average increment in Tₘ per modification for thyclotide was found to be around 4° C. Desirably, the fully modified thyclotide, 3d, could bind to complementary DNA with extraordinary high binding affinity and Tₘ of its corresponding duplex was found to be 77° C., which is 35° C. higher than that of aegPNA-DNA duplex (entry 5). Although the stabilizing effect of tetrahydrofuran for PNA-DNA duplex is slightly lower than that of cyclopentane (3d vs 9 and 2a, Table 2), it strikes a balance between water solubility and binding properties and, therefore the combination of those two backbone modifications could be tailor-made to meet the specific requirements.

TABLE 2
Melting Temperature Data for PNA-DNA Complexes

| entry^{a} | sequence | Tₘ^{b} (° C.) | ΔTm^{c} (° C.) |
|---|---|---|---|
| 1 | GATGTGATA-AEEA (1a) | 42.0 | — |
| 2 | GATGT*GATA-AEEA (3a) | 44.7 | 2.7 |
| 3 | GAT*GTGAT*A-AEEA (3b) | 49.7 | 7.7 |
| 4 | GAT*GT*GAT*A-AEEA (3c) | 54.9 | 12.9 |
| 5 | G*A*T*G*T*G*A*T*A*- | 77.0 | 35.0 |
| | AEEA (3d) | | |
| 6 | G*A*T*G#T#G#A*T*A* | 84.5 | 42.5 |
| | AEEA (9) | | |
| 7 | G#A#T#G#T#G#A#T#A#- | 93.7 | 51.7 |
| | AEEA (2a) | | |

### Example 4

This example demonstrates the sequence specificity of the inventive oligomer, in accordance with an aspect of the invention.

The sequence specificity of thyclotide 3d was examined by determining the change in Tₘ of a PNA-DNA duplex when a single base mismatch is present. The thermal denaturation studies showed that the difference in the melting temperature between the fully matched PNA-DNA duplex of thyclotide 3d and the duplex with a single base mismatch (ΔTₘ) is significantly higher than that of the corresponding aegPNA and slightly lower to these of tcypPNA 2a and mixPNA 9 (Table 3). The large differences in melting temperature between matched PNA-DNA duplex and mismatch PNA-DNA of thyclotide 3d allows one to control hybridization conditions to exclude the formation of mismatched PNA-DNA by adjust the temperature. Such enhanced single base pair mismatch-discrimination capability could make thyclotide 3d become a powerful tool for the detection of point-mutations and single nucleotide polymorphisms (SNPs).

TABLE 3
Discrimination of Single Base Mismatches and Improvements in Thyclotide

| | | ΔTm (Tₘ)^{b} | | |
|---|---|---|---|---|
| entry | sequence | TT mismatch | TC mismatch | TG mismatch |
| 1 | GATG [symbol] GATA-AEEA (1a) | 28.2 (−13.8) | 22.9 (−19.2) | 25.4 (−16.6) |
| 2 | G*A*T*G* [symbol] *G*A*T*A* | 58.2 (−18.8) | 50.7 (−26.2) | 55.6 (−21.4) |
| | AEEA (3d) | | | |
| 3 | G*A*T*G* [symbol] *G*A*T*A*- | 65.0 (−19.5) | 57.7 (−27.4) | 64.2 (−20.3) |
| | AEEA (9) | | | |
| 4 | G*A*T*G* [symbol] *GAA*T*A*- | 70.9 (−22.8) | 63.1 (−30.6) | 73.1 (−20.6) |
| | AEEA (2a) | | | |

### Example 5

This example demonstrates the effect of tetrahydrofuran on the conformation of PNA.

The CD spectra of thyclotides 3a-3d were measured. The unmodified PNA 1a does not show any distinct peaks suggesting an unstructured non-helical conformation (Sforza, S. et al., Eur. J. Org. Chem., 197 (1999)). However, in the case of thyclotides 3a-3d, distinct distinctive positive peaks around 220 and 269 nm and negative peaks around 204 and 247 nm were noticed. These distinctive cotton effects suggest a right-handed helical conformation similar to the B-form DNA helix (Kypr, J.; Kejnovská, I. et al., Nucleic Acids Res. 37: 1713 (2009)). The amplitude of the cotton effects increases as more tetrahydrofuran modifications are introduced. Although the amplitude of CD signals for thyclotides are smaller than these of mixPNA 9 and tcypPNA 2a, these CD data indicate that introduction of tetrahydrofuran modification could preorganize PNAs in right-handed helix forms to some extent, which most likely accounts for their enhanced binding affinity to the complementary DNA.

### Example 6

This example demonstrates the cell uptake of an inventive oligomer as compared with an aminoethylglycine (aeg) PNA, in accordance with an aspect of the invention.

Flow cytometry was performed under the following conditions: 5 μM of AEG-PNA or 5 μM thyclotide; 3 hours incubation time. The structures of the AEGPNA and the thyclotide are shown in FIG. 5.

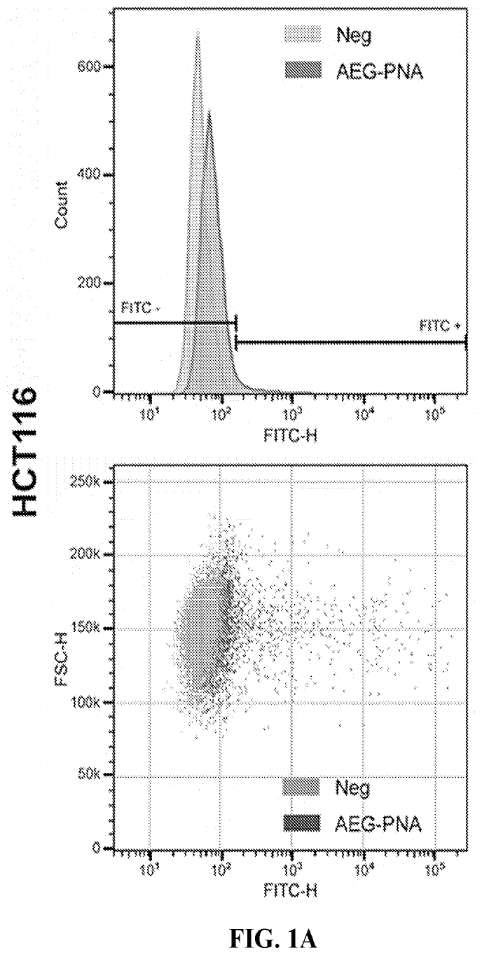

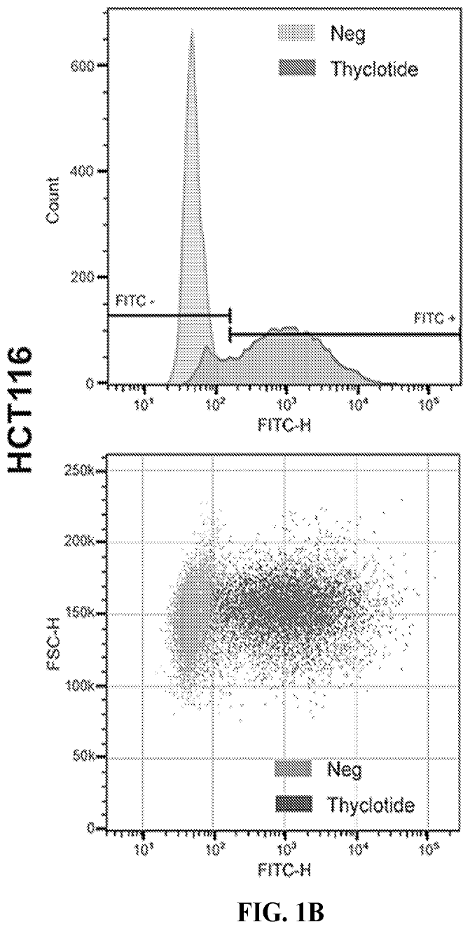

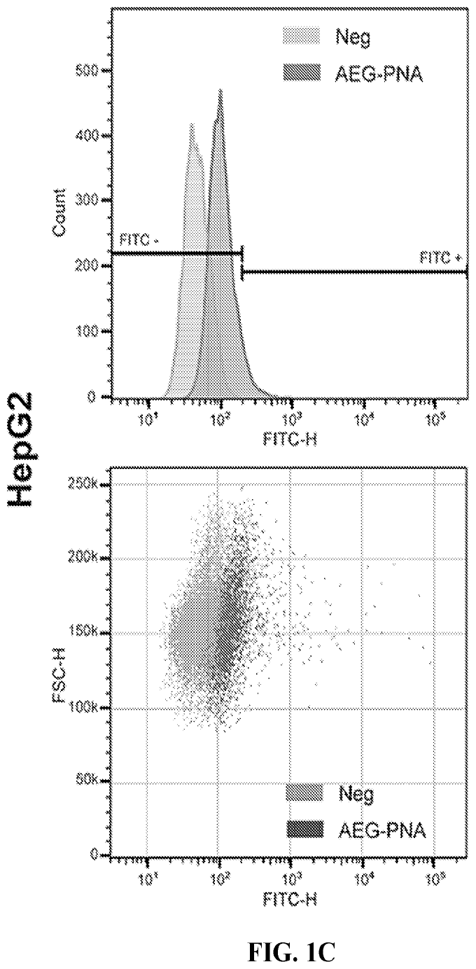

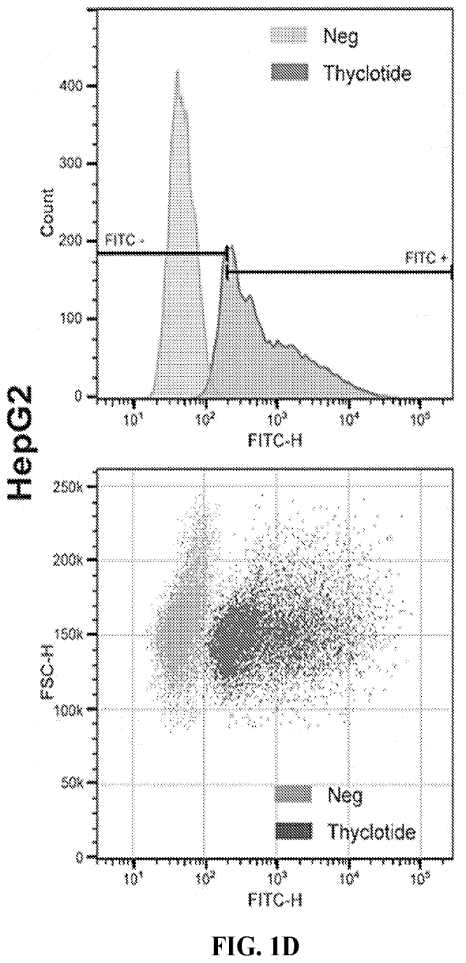

Cell uptake experiment was realized in two different cancer cell lines. Both HCT116 and HepG2 cell lines show a significantly higher number of fluorescent cells following incubation with thyclotide compared to cells incubated with regular AEG-PNA (P<0.0001 and P=0.0022 respectively). Only 3 hours after treatment, close to 80% of cells were already positive for thyclotide. For HCT116 cells, the cell counts and scatter graphs for AEG-PNA and for thyclotide are shown in FIGS. 1A and 1B, respectively. For Hep 62 cells, the cell counts and scatter graphs for AEG-PNA and for thyclotide are shown in FIGS. 1C and 1D, respectively.

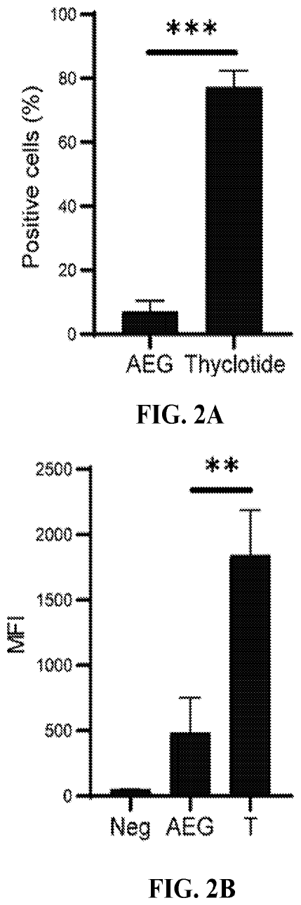

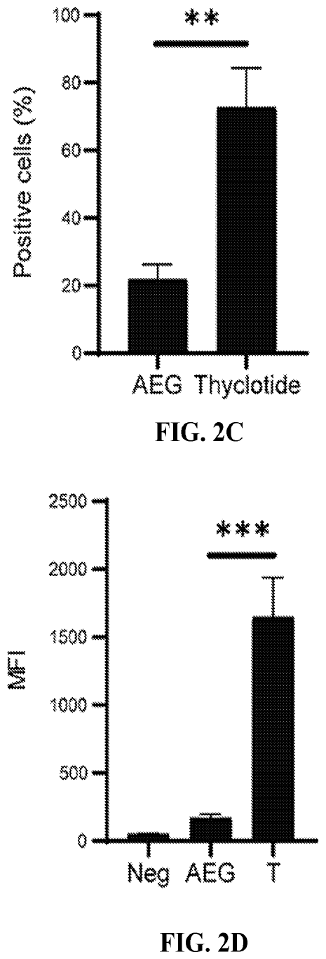

In addition, the mean of fluorescence (MFI) is much higher after incubation with thyclotide (T) compared to AEG-PNA treatment in both cell lines. HCT116 cells display a significant 4-fold increase in MFI (P=0.0058) and HepG2 cells a 10-fold increase in MFI (P=0.0009). For HCT116 cells, the percent of positive cells and mean fluorescence intensity (MFI) for AEG-PNA and for thyclotide are shown in FIGS. 2A and 2B, respectively. For Hep62 cells, the percent of positive cells and mean fluorescence intensity (MFI) for AEG-PNA and for thyclotide are shown in FIGS. 2C and 2D, respectively.

### Example 7

This example demonstrates the uptake of thyclotide by HCT116 cells as observed by microscopy, in accordance with an aspect of the invention.

5 μM of AEG-PNA or 5 μM thyclotide were incubated for 3 hours with HCT116 cells. The structures of the AEG-PNA or 5 μM thyclotide are shown in FIG. 5, both of which have a fluorescein label.

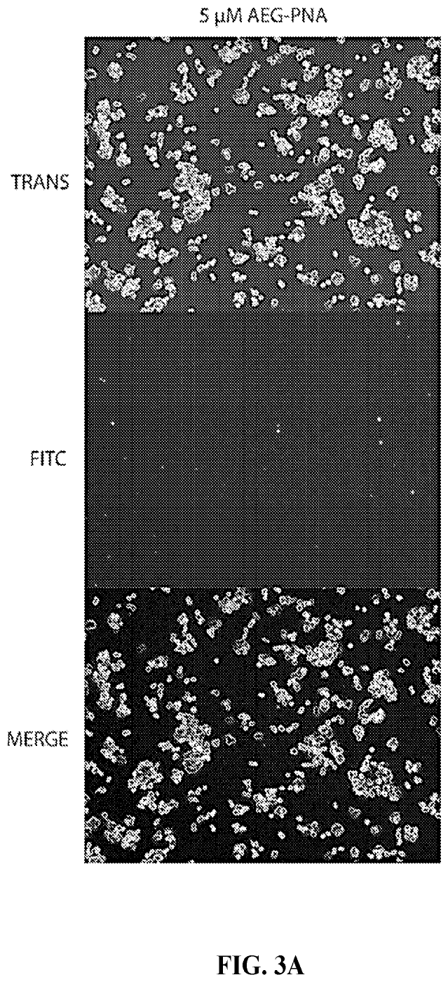

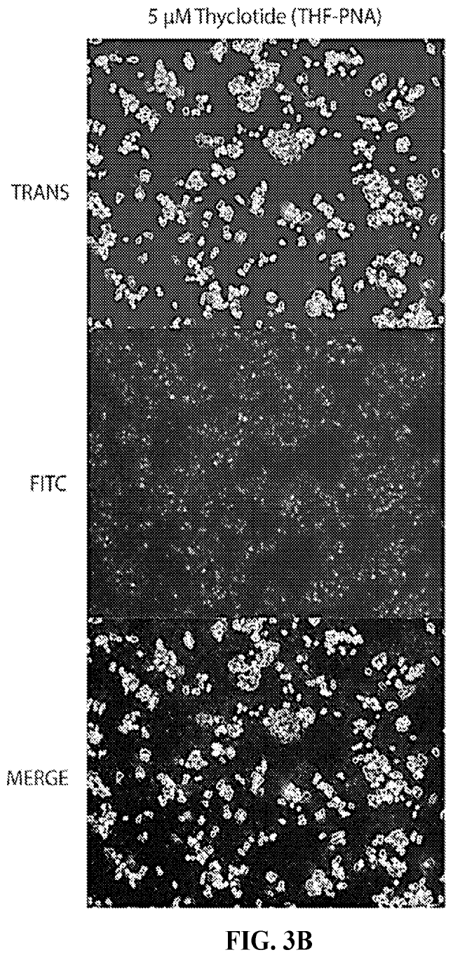

The microscopic images are shown in FIGS. 3A and 3B. As is apparent from the images in FIGS. 3A and 3B, 3 hours after the treatment, only HCT116 cells incubated with thyclotide display a strong fluorescence.

### Example 8

This example demonstrates the effect of an exemplary thyclotide on the expression of microRNA-21 (miR-21) in accordance with an aspect of the invention.

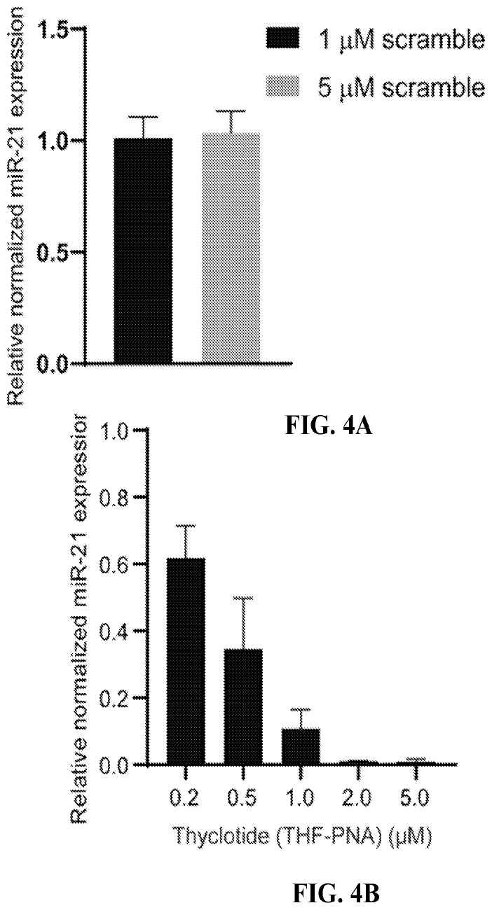

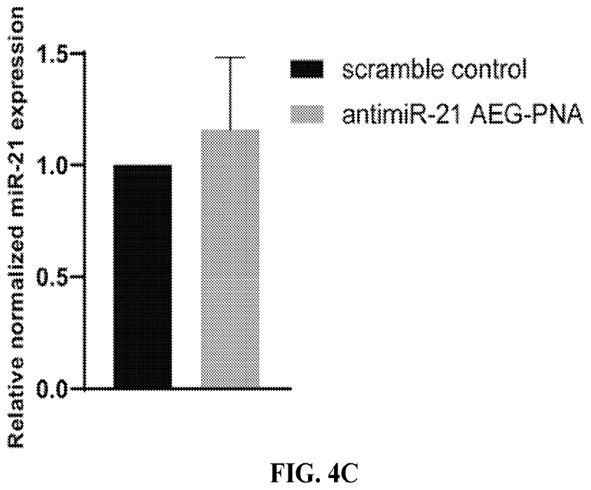

A thyclotide with a scrambled sequence, not targeting miR-21, was used to treat cells at a concentration of 1 and 5 μM. Expression was normalized to non-treated cells using both miR-25 and miR-93 as endogenous controls. The result is shown in FIG. 4A. Whatever the concentration, the scrambled thyclotide has no effect on miR-21 expression.

Different concentrations of antimiR-21 thyclotide were used to treat cells for 48 hours before assessing the expression of miR-21 by RT-qPCR. Expression of miR-21 was normalized to scrambled control-treated cells using both miR-25 and miR-93 as endogenous controls. The results are shown in FIG. 4B, which demonstrate that 2 μM of antimiR-21 thyclotide can totally inhibit miR-21 expression.

1 μM of regular antimiR-21 AEG-PNA was used to treat cells in the same way as antimiR-21 thyclotide previously. Expression of miR-21 was normalized to scrambled control-treated cells using both miR-25 and miR-93 as endogenous controls. The results are shown in FIG. 4C, which show that the antimiR-21 AEG-PNA had no effect on expression of miR-21.

All experiments were independently repeated at least 3 times.

### Example 9

This example demonstrates the difference in polarity of an inventive oligomer as compared with AEG-PNA, a mixed AEG and cyclopentyl PNA, and cyclopentyl-containing PNA determined by HPLC, in accordance with an aspect of the invention.

Waters (Milford, Mass., USA) XBridge™ C18 (4.6×250 mm, 5 μm) columns were used in conjunction with Solvents A and B for purity checking at room temperature. Solvent A was 0.1% TFA in water and Solvent B consisted of 90% acetonitrile in water.

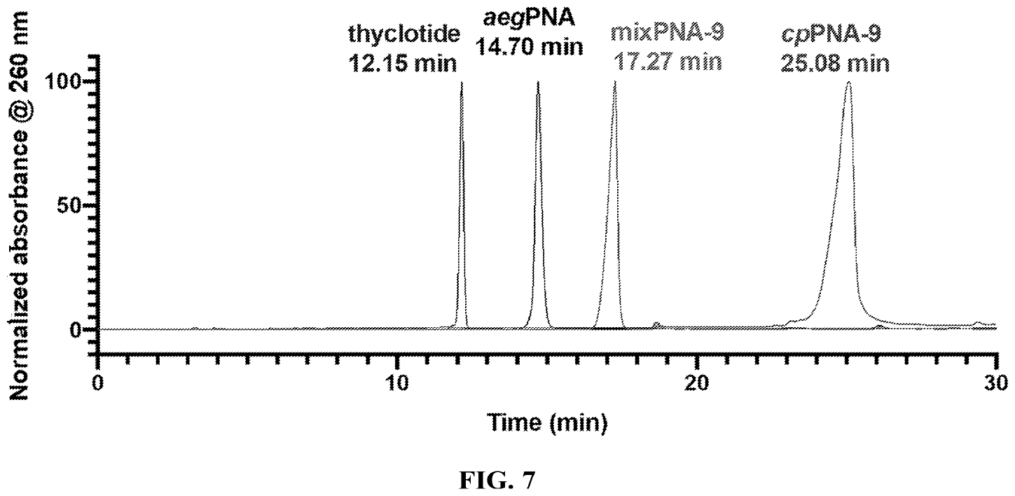

HPLC conditions: the column was equilibrated with 100% A for 15 min prior to run, a linear gradient from 0% B to 40% B over 30 min, a linear gradient to 100% B over 1 min, 100% B for 4 min, and a linear gradient to 0% B over 1 min. A representative HPLC chromatogram is shown in FIG. 7. As is apparent from FIG. 7, the inventive oligomer is significantly less polar than the AEG-PNA, a mixed AEG and cyclopentyl PNA, and cyclopentyl-containing PNA.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An oligomer comprising (a) from about 8 to about 25 monomer units of formula (I): 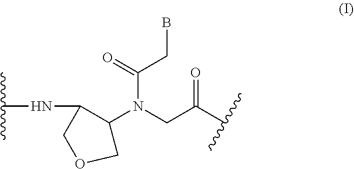
and
(b) from 0 to about 24 monomer units of formula (II): 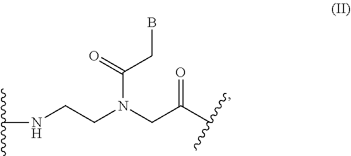
wherein B is a nucleobase, and wherein B can be the same or different at each occurrence,
or a pharmaceutically acceptable salt thereof.

2. The oligomer or salt of claim 1, wherein B is adenine, thymine, uracil, guanine, or cytosine or is a nucleobase of a non-natural nucleotide.

3. The oligomer or salt of claim 1, wherein the oligonucleotide comprises a sum of from about 12 to about 25 monomer units of formula (I) and formula (II).

4. (canceled)

5. The oligomer or salt of claim 1, wherein the oligomer does not comprise a monomer unit of formula (II).

6. The oligomer or salt of claim 1, wherein the monomer unit of formula (I) has two chiral carbon atoms, wherein the two chiral carbon atoms of at least one monomer unit of formula (I) have an (R,R) configuration or an (S,S) configuration.

7. The oligomer or salt of claim 1, wherein the oligomer further comprises one or more monomer units of formula (III): 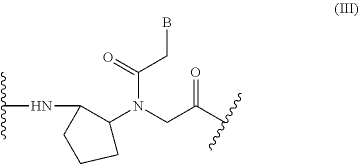
wherein B is a nucleobase, and wherein B can be the same or different at each occurrence.

8. The oligomer or salt of claim 7, wherein the oligonucleotide comprises a sum of from about 12 to about 25 monomer units of formula (I), formula (II), and formula (III).

9. (canceled)

10. The oligomer or salt of claim 1, wherein the oligomer has an amino terminus and a carboxy terminus, wherein the amino terminus is —NHR¹ wherein R¹ is H or a linker-attached group selected from dyes, radioimaging moieties, chelating moieties, nanoparticles, cytotoxic agents, and a second oligomer comprising (a) from about 8 to about 25 monomer units of formula (I) and (b) from 0 to about 24 monomer units of formula (II), wherein the monomer unit of formula (I) in the second oligomer has two chiral carbon atoms, wherein the two chiral carbon atoms of at least one monomer unit of formula (I) in the second oligomer have an (S,S) configuration or an (R,R) configuration.

11. The oligomer or salt of claim 10, wherein R¹ comprises a dye selected from fluorescein, Cy2, Cy3, Cy3.5, Cy5, Cy5.5, Cy7, and Cy7.5.

12. The oligomer or salt of claim 10, wherein R¹ comprises a chelating agent selected from 1,4,7-triazacyclononane-N,N′,N″-triacetic acid, 1,4,7,10-tetrazacyclononane-N,N′,N″-triacetic acid, triethylenetetramine, diethylenetetramine pentaacetic acid, and hydrazinonicotinamide.

13. The oligomer or salt of claim 10, wherein R¹ comprises a radioimaging agent selected from 1,4,7-triazacyclononane-N,N′,N″-triacetic acid, 1, 4, 7,10-tetrazacyclononane-N,N′,N″-triacetic acid, triethylenetetramine, diethylenetetramine pentaacetic acid, and hydrazinonicotinamide in combination with one or more of ¹⁸F-AlF, ⁶⁰Cu, ⁶¹Cu, ⁶²Cu, ⁶⁴Cu, ⁶⁷Cu, ⁶⁸Ga, ⁸⁶Y, ⁸⁹Zr, ¹¹¹In, ⁹⁹mTc, ¹⁸⁶Re, ¹⁸⁸Re, Gd³⁺, or Mn²⁺.

14. The oligomer or salt of claim 10, wherein the second oligomer comprises a sum of from about 12 to about 25 monomer units of formula (I) and formula (II).

15. The oligomer or salt of claim 10, wherein the linker is —C(═O)CH₂(OCH₂CH₂)ₘ—NH— wherein m is an integer of from 1 to about 10 or —(CH₂CH₂O)ₚ wherein p is an integer of from 1 to about 20.

16. The oligomer or salt of claim 10, wherein the carboxy terminus is —C(═O)—R² wherein R² is selected from OH, —NHC(═O)(CH₂CH₂O)ₙCH₂CONH₂, and —NH(CH₂CH₂)ₒH, wherein n and o are independently integers of from 1 to about 10.

17. The oligomer or salt of claim 1, wherein the oligomer comprises a monomer sequence that is complementary to a target nucleotide sequence.

18-23. (canceled)

24. The oligomer or salt of claim 1, wherein the oligomer has the sequence AGTCTGATAAGCTA (SEQ ID NO: 1).

25. A pharmaceutical composition comprising the oligomer or salt of claim 1 and a pharmaceutically acceptable carrier.

26. A compound of formula (IV): 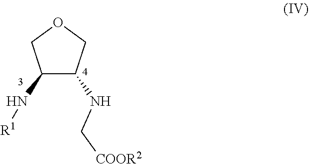
wherein R¹ is a nitrogen protecting group and wherein R² is selected from H, C₁-C₁₀ alkyl, C₆-C₁₀ aryl, and C₁-C₁₀ alkyl-C₆-C₁₀ aryl, wherein the configuration at positions 3 and 4 of the tetrahydrofuran ring is (3R,4R) or (3S,4S).

27-28. (canceled)

29. A compound of formula (V): 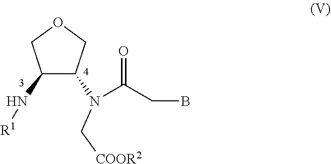
wherein R¹ is a nitrogen protecting group, wherein R² is selected from H, C₁-C₁₀ alkyl, C₆-C₁₀ aryl, and C₁-C₁₀ alkyl-C₆-C₁₀ aryl, and wherein B is an optionally protected nucleobase, wherein the configuration at positions 3 and 4 of the tetrahydrofuran ring is (3R,4R) or (3S,4S).

30. The compound of claim 29, wherein B is selected from adenine, 4-(benzhydryloxycarbonyl)adenine, guanosine, 2-(benzhydryloxycarbonyl)guanosine, cytosine, 4-(benzhydryloxycarbonyl)cytosine, thymine, and uracil.

31-32. (canceled)
